(19) United States
(12) Patent Application Publication
(10) Pub. No.: US 2021/0145882 A1
(43) Pub. Date: May 20, 2021

(54) METHODS FOR ADOPTIVE CELL THERAPY TARGETING ROR1

(71) Applicants: FRED HUTCHINSON CANCER RESEARCH CENTER, Seattle, WA (US); UNIVERSITY OF WASHINGTON, Seattle, WA (US)

(72) Inventors: David G. MALONEY, Issaquah, WA (US); Jennifer SPECHT, Lynnwood, WA (US); Stanley R. RIDDELL, Sammamish, WA (US)

(21) Appl. No.: 17/047,360

(22) PCT Filed: Apr. 12, 2019
(86) PCT No.: PCT/US2019/027371

§ 371 Date: Oct. 13, 2020

Related U.S. Application Data
(60) Provisional application No. 62/657,642, filed on Apr. 13, 2018.

(51) Int. Cl. A61K 35/17; C07K 16/28; C07K 14/705; C07K 14/725; A61K 31/675; A61K 31/7076; A61K 31/282; A61P 35/00; A61P 35/04; A61K 45/06

(52) U.S. Cl. CPC A61K 35/17; C07K 2317/53; C07K 14/70578; C07K 14/7051; C07K 14/70517; C07K 14/70521; A61K 31/675; A61K 31/7076; A61K 31/282; A61P 35/00; A61P 35/04; A61K 45/06; C07K 2317/56; C07K 2317/565; C07K 2319/03; C07K 2319/33; C07K 2317/622; C07K 16/2803

(57) ABSTRACT

The present disclosure provides methods and compositions for treating cancers using modified immune cells that express a ROR1-specific binding protein according to certain treatment protocols including, for example, dosing regimens, infusion schedules, and patient selection criteria. In certain embodiments, presently disclosed methods and compositions are useful for treating solid cancers and/or hematological malignancies (e.g., triple-negative breast cancer (TBNC), non-small cell lung cancer (NSCLC), mantle cell lymphoma (MCL), acute lymphoblastic leukemia (ALL), or chronic lymphocytic leukemia (CLL)), wherein the methods comprise administering modified T cells that target a ROR1 antigen.

### STATEMENT OF GOVERNMENT INTEREST

This invention was made with government support under CA114536 awarded by the National Institutes of Health. The government has certain rights in the invention.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 360056_462 WO_SEQUENCE_LISTING.txt. The text file is 27.4 KB, was created on Apr. 11, 2019, and is being submitted electronically via EFS-Web.

### BACKGROUND

Adoptive transfer of genetically modified T cells has emerged as a potent therapy for various malignancies. The most widely employed strategy has been infusion of patient-derived T cells expressing chimeric antigen receptors (CARs) targeting tumor associated antigens. This approach has numerous theoretical advantages, including the ability to target T cells to any cell surface antigen, circumvent loss of major histocompatibility complex as a tumor escape mechanism, and employ a single vector construct to treat any patient, regardless of human leukocyte antigen haplotype. However, adoptive cell therapies are still developing, and new regimens for cell therapies are needed. The presently disclosed embodiments address these needs and provide other related advantages.

### BRIEF DESCRIPTION OF THE DRAWINGS

FIG. 1 shows a schematic of a cell-surface expressed chimeric antigen receptor (CAR) of the present disclosure, comprising an antigen-binding scFv domain that targets the ROR1 antigen, an extracellular spacer domain, a transmembrane domain, and 4-1BB and CD3ζ intracellular signaling components.

FIG. 2 shows a treatment schema according to the present disclosure. The treatment protocol investigated the safety and efficacy of engineered T cells expressing a ROR1-specific CAR for treating ROR1+ hematological or solid cancers: mantle cell lymphoma (MCL); acute lymphoblastic leukemia (ALL); chronic lymphocytic leukemia (CLL); non-small cell lung cancer (NSCLC); or triple-negative breast cancer (TBNC).

FIG. 3 shows hematoxylin and eosin stain (H&E) of a pre-treatment tumor biopsy from patient X566 with membranous ROR1 expression at 10× (L) and 40× (R).

FIGS. 4A-4L show serum cytokine profiles (pg/mL) from the indicated patients taken at various time points before (pre-0) and after (1 hr-28 days) ROR1 CAR-T cell infusion. Cytokine profiles were determined using a Luminex cytokine assay. (A, G) IFN-γ. (B, H) IL-6. (C, I) IL-8. (D, J) IL-7. (E, K) IL-15. (F, L) IL-10.

FIGS. 5A-5J show persistence of ROR1 CAR-T cells of the present disclosure in the indicated patients following infusion. Cells were administered at Day 0 (shown with an arrow), and the following data were measured at the indicated timepoints from patient PBMCs: percentage of T cells that expressed the transgene transduction marker (5A and 5F=CD4+, 5B and 5G=CD8+); and counts of transgene-expressing cells per microliter present in the samples (5C and 5H=CD4+, 5D and 5I=CD8+). For the data shown in FIGS. 5A-5D and 5F-5I, aliquots were stained with mAbs specific for CD45, CD3, CD4, CD8, and Eribitux-SA-PE. FIGS. 5E and 5J show data from a flap EF1 alpha-specific qPCR assay used to examine PBMCs for the presence of transgene vector-specific DNA sequences.

FIGS. 6A-6D show the percentage of ROR1 CAR-T cells expressing T cell activation and exhaustion markers in the infusion product (black circles) and on Day +14 in vivo (open squares). Expression of the indicated cell surface markers is shown on (A, C) CD4+ CAR-T cells and (B, D) CD8+ CAR-T cells.

FIGS. 7A-7E show multiplex immunohistochemistry (IHC) staining on a patient tumor biopsy pre- (A, B) and post-treatment (C, D) with the ROR1 CAR-T cell product. At left of each figure is the sample at low magnification. At right of each figure is the sample at high magnification. Staining with primary antibodies (anti-CD3, -COX2, -CD206, -VISTA/B7-H5, -CD163) is as shown in the figure keys. Nuclear staining (DAPI) as a counterstain. Imaging was performed using the Vectra 3.0 platform. (E) HALO-based quantification depicting log 10 fold-change of cell densities across the entire sample.

### DETAILED DESCRIPTION

The present disclosure provides reagents and methods for treating cancer using modified immune cells (e.g., T cells comprising a CAR or a TCR) according to treatment protocols including, for example, dosing regimens, infusion schedules, and patient selection criteria. In certain embodiments, presently disclosed methods and compositions are useful for treating solid cancers and/or hematological malignancies (e.g., triple-negative breast cancer (TBNC), non-small cell lung cancer (NSCLC), mantle cell lymphoma (MCL), acute lymphoblastic leukemia (ALL), or chronic lymphocytic leukemia (CLL)), wherein the methods comprise administering modified T cells that target a ROR1 antigen.

Prior to setting forth this disclosure in more detail, it may be helpful to an understanding thereof to provide definitions of certain terms to be used herein. Additional definitions are set forth throughout this disclosure.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. As used herein, the term “about” means±20% of the indicated range, value, or structure, unless otherwise indicated. It should be understood that the terms “a” and “an” as used herein refer to “one or more” of the enumerated components. The use of the alternative (e.g., “or”) should be understood to mean either one, both, or any combination thereof of the alternatives. As used herein, the terms “include,” “have” and “comprise” are used synonymously, which terms and variants thereof are intended to be construed as non-limiting.

“Optional” or “optionally” means that the subsequently described element, component, event, or circumstance may or may not occur, and that the description includes instances in which the element, component, event, or circumstance occurs and instances in which they do not.

In addition, it should be understood that the individual constructs, or groups of constructs, derived from the various combinations of the structures and subunits described herein, are disclosed by the present application to the same extent as if each construct or group of constructs was set forth individually. Thus, selection of particular structures or particular subunits is within the scope of the present disclosure.

The term “consisting essentially of” is not equivalent to “comprising” and refers to the specified materials or steps that do not materially affect the basic characteristics of a claimed invention. For example, a protein domain, region, or module (e.g., a binding domain, hinge region, or linker) or a protein (which may have one or more domains, regions, or modules) “consists essentially of” a particular amino acid sequence when the amino acid sequence of a domain, region, module, or protein includes extensions, deletions, mutations, or a combination thereof (e.g., amino acids at the amino- or carboxy-terminus or between domains) that, in combination, contribute to at most 20% (e.g., at most 15%, 10%, 8%, 6%, 5%, 4%, 3%, 2% or 1%) of the length of a domain, region, module, or protein and do not substantially affect (i.e., do not reduce the activity by more than 50%, such as no more than 40%, 30%, 25%, 20%, 15%, 10%, 5%, or 1%) the activity of the domain(s), region(s), module(s), or protein (e.g., the target binding affinity of a binding protein).

As used herein, “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refer to compounds that have the same basic chemical structure as a naturally occurring amino acid, i.e., an α-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refer to chemical compounds that have a structure that is different from the general chemical structure of an amino acid, but that functions in a manner similar to a naturally occurring amino acid.

As used herein, “mutation” refers to a change in the sequence of a nucleic acid molecule or polypeptide molecule as compared to a reference or wild-type nucleic acid molecule or polypeptide molecule, respectively. A mutation can result in several different types of change in sequence, including substitution, insertion or deletion of nucleotide(s) or amino acid(s).

A “conservative substitution” refers to amino acid substitutions that do not significantly affect or alter binding characteristics of a particular protein. Generally, conservative substitutions are ones in which a substituted amino acid residue is replaced with an amino acid residue having a similar side chain. Conservative substitutions include a substitution found in one of the following groups: Group 1: Alanine (Ala or A), Glycine (Gly or G), Serine (Ser or S), Threonine (Thr or T); Group 2: Aspartic acid (Asp or D), Glutamic acid (Glu or Z); Group 3: Asparagine (Asn or N), Glutamine (Gln or Q); Group 4: Arginine (Arg or R), Lysine (Lys or K), Histidine (His or H); Group 5: Isoleucine (Ile or I), Leucine (Leu or L), Methionine (Met or M), Valine (Val or V); and Group 6: Phenylalanine (Phe or F), Tyrosine (Tyr or Y), Tryptophan (Trp or W). Additionally or alternatively, amino acids can be grouped into conservative substitution groups by similar function, chemical structure, or composition (e.g., acidic, basic, aliphatic, aromatic, or sulfur-containing). For example, an aliphatic grouping may include, for purposes of substitution, Gly, Ala, Val, Leu, and Ile. Other conservative substitutions groups include: sulfur-containing: Met and Cysteine (Cys or C); acidic: Asp, Glu, Asn, and Gln; small aliphatic, nonpolar or slightly polar residues: Ala, Ser, Thr, Pro, and Gly; polar, negatively charged residues and their amides: Asp, Asn, Glu, and Gln; polar, positively charged residues: His, Arg, and Lys; large aliphatic, nonpolar residues: Met, Leu, Ile, Val, and Cys; and large aromatic residues: Phe, Tyr, and Trp. Additional information can be found in Creighton (1984) Proteins, W.H. Freeman and Company.

As used herein, “protein” or “polypeptide” refers to a polymer of amino acid residues. Proteins apply to naturally occurring amino acid polymers, as well as to amino acid polymers in which one or more amino acid residue is an artificial chemical mimetic of a corresponding naturally occurring amino acid and non-naturally occurring amino acid polymers.

As used herein, “fusion protein” refers to a protein that, in a single chain, has at least two distinct domains, wherein the domains are not naturally found together in a protein. A polynucleotide encoding a fusion protein may be constructed using PCR, recombinantly engineered, or the like, or such fusion proteins can be synthesized. A fusion protein may further contain other components, such as a tag, a linker, or a transduction marker. In certain embodiments, a fusion protein comprises a binding protein that is expressed or produced by a host cell (e.g., a T cell) locates to a cell surface, where the binding protein is anchored to the cell membrane (e.g., via a transmembrane domain) and comprises an extracellular portion (e.g., containing a binding domain) and an intracellular portion (e.g., containing a effector domain, effector domain, co-stimulatory domain or combinations thereof).

“Nucleic acid molecule” or “polynucleotide” refers to a polymeric compound including covalently linked nucleotides, which can be made up of natural subunits (e.g., purine or pyrimidine bases) or non-natural subunits (e.g., morpholine ring). Purine bases include adenine, guanine, hypoxanthine, and xanthine, and pyrimidine bases include uracil, thymine, and cytosine. Nucleic acid molecules include polyribonucleic acid (RNA), polydeoxyribonucleic acid (DNA), which includes cDNA, genomic DNA, and synthetic DNA, either of which may be single or double stranded. If single stranded, the nucleic acid molecule may be the coding strand or non-coding (anti-sense) strand. A nucleic acid molecule encoding an amino acid sequence includes all nucleotide sequences that encode the same amino acid sequence. Some versions of the nucleotide sequences may also include intron(s) to the extent that the intron(s) would be removed through co- or post-transcriptional mechanisms. In other words, different nucleotide sequences may encode the same amino acid sequence as the result of the redundancy or degeneracy of the genetic code, or by splicing.

“Percent sequence identity” refers to a relationship between two or more sequences, as determined by comparing the sequences. Preferred methods to determine sequence identity are designed to give the best match between the sequences being compared. For example, the sequences are aligned for optimal comparison purposes (e.g., gaps can be introduced in one or both of a first and a second amino acid or nucleic acid sequence for optimal alignment). Further, non-homologous sequences may be disregarded for comparison purposes. The percent sequence identity referenced herein is calculated over the length of the reference sequence, unless indicated otherwise. Methods to determine sequence identity and similarity can be found in publicly available computer programs. Sequence alignments and percent identity calculations may be performed using a BLAST program (e.g., BLAST 2.0, BLASTP, BLASTN, or BLASTX). The mathematical algorithm used in the BLAST programs can be found in Altschul et al., Nucleic Acids Res. 25:3389-3402, 1997. Within the context of this disclosure, it will be understood that where sequence analysis software is used for analysis, the results of the analysis are based on the “default values” of the program referenced. “Default values” mean any set of values or parameters which originally load with the software when first initialized.

Variants of nucleic acid molecules of this disclosure are also contemplated. Variant nucleic acid molecules include those that are at least 70%, 75%, 80%, 85%, 90%, and preferably 95%, 96%, 97%, 98%, 99%, or 99.9% identical a nucleic acid molecule of a defined or reference polynucleotide as described herein, or that hybridize to a polynucleotide under stringent hybridization conditions of 0.015M sodium chloride, 0.0015M sodium citrate at about 65-68° C. or 0.015M sodium chloride, 0.0015M sodium citrate, and 50% formamide at about 42° C. Nucleic acid molecule variants retain the capacity to encode a binding protein or a binding domain thereof having a functionality described herein, such as specifically binding a target molecule.

A “functional variant” refers to a polypeptide or polynucleotide that is structurally similar or substantially structurally similar to a parent or reference compound of this disclosure, but differs slightly in composition (e.g., one base, atom or functional group is different, added, or removed), such that the polypeptide or encoded polypeptide is capable of performing at least one function of the encoded parent polypeptide with at least 50% efficiency, preferably at least 55%, 60%, 70%, 75%, 80%, 85%, 90%, 95% or 100% level of activity of the parent polypeptide. In other words, a functional variant of a polypeptide or encoded polypeptide of this disclosure has “similar binding,” “similar affinity” or “similar activity” when the functional variant displays no more than a 50% reduction in performance in a selected assay as compared to the parent or reference polypeptide, such as an assay for measuring binding affinity (e.g., Biacore® or tetramer staining measuring an association (Ka) or a dissociation (KD) constant).

As used herein, a “functional portion” or “functional fragment” refers to a polypeptide or polynucleotide that comprises only a domain, portion or fragment of a parent or reference compound, and the polypeptide or encoded polypeptide retains at least 50% activity associated with the domain, portion or fragment of the parent or reference compound, preferably at least 55%, 60%, 70%, 75%, 80%, 85%, 90%, 95% or 100% level of activity of the parent polypeptide, or provides a biological benefit (e.g., effector function). A “functional portion” or “functional fragment” of a polypeptide or encoded polypeptide of this disclosure has “similar binding” or “similar activity” when the functional portion or fragment displays no more than a 50% reduction in performance in a selected assay as compared to the parent or reference polypeptide (preferably no more than 20% or 10%, or no more than a log difference as compared to the parent or reference with regard to affinity), such as an assay for measuring binding affinity or measuring effector function (e.g., cytokine release).

The term “isolated” means that the material is removed from its original environment (e.g., the natural environment if it is naturally occurring). For example, a naturally occurring nucleic acid or polypeptide present in a living animal is not isolated, but the same nucleic acid or polypeptide, separated from some or all of the co-existing materials in the natural system, is isolated. Such nucleic acid could be part of a vector and/or such nucleic acid or polypeptide could be part of a composition (e.g., a cell lysate), and still be isolated in that such vector or composition is not part of the natural environment for the nucleic acid or polypeptide. The term “gene” means the segment of DNA involved in producing a polypeptide chain; it includes regions preceding and following the coding region (“leader and trailer”) as well as intervening sequences (introns) between individual coding segments (exons).

As used herein, the term “endogenous” or “native” refers to a polynucleotide, gene, protein, compound, molecule, or activity that is normally present in a host cell or a subject.

As used herein, “heterologous” or “non-endogenous” or “exogenous” refers to any gene, protein, compound, nucleic acid molecule, or activity that is not native to a host cell or a subject, or any gene, protein, compound, nucleic acid molecule, or activity native to a host cell or a subject that has been altered. Heterologous, non-endogenous, or exogenous includes genes, proteins, compounds, or nucleic acid molecules that have been mutated or otherwise altered such that the structure, activity, or both is different as between the native and altered genes, proteins, compounds, or nucleic acid molecules. In certain embodiments, heterologous, non-endogenous, or exogenous genes, proteins, or nucleic acid molecules (e.g., receptors, ligands, etc.) may not be endogenous to a host cell or a subject, but instead nucleic acids encoding such genes, proteins, or nucleic acid molecules may have been added to a host cell by conjugation, transformation, transfection, electroporation, or the like, wherein the added nucleic acid molecule may integrate into a host cell genome or can exist as extra-chromosomal genetic material (e.g., as a plasmid or other self-replicating vector). The term “homologous” or “homolog” refers to a gene, protein, compound, nucleic acid molecule, or activity found in or derived from a host cell, species, or strain. For example, a heterologous or exogenous polynucleotide or gene encoding a polypeptide may be homologous to a native polynucleotide or gene and encode a homologous polypeptide or activity, but the polynucleotide or polypeptide may have an altered structure, sequence, expression level, or any combination thereof. A non-endogenous polynucleotide or gene, as well as the encoded polypeptide or activity, may be from the same species, a different species, or a combination thereof.

The term “expression”, as used herein, refers to the process by which a polypeptide is produced based on the encoding sequence of a nucleic acid molecule, such as a gene. The process may include transcription, post-transcriptional control, post-transcriptional modification, translation, post-translational control, post-translational modification, or any combination thereof. An expressed nucleic acid molecule is typically operably linked to an expression control sequence (e.g., a promoter).

The term “operably linked” refers to the association of two or more nucleic acid molecules on a single nucleic acid fragment so that the function of one is affected by the other. For example, a promoter is operably linked with a coding sequence when it is capable of affecting the expression of that coding sequence (i.e., the coding sequence is under the transcriptional control of the promoter). “Unlinked” means that the associated genetic elements are not closely associated with one another and the function of one does not affect the other.

As used herein, “expression vector” refers to a DNA construct containing a nucleic acid molecule that is operably linked to a suitable control sequence capable of effecting the expression of the nucleic acid molecule in a suitable host. Such control sequences include a promoter to effect transcription, an optional operator sequence to control such transcription, a sequence encoding suitable mRNA ribosome binding sites, and sequences which control termination of transcription and translation. The vector may be a plasmid, a phage particle, a virus, or simply a potential genomic insert. Once transformed into a suitable host, the vector may replicate and function independently of the host genome, or may, in some instances, integrate into the genome itself. In the present specification, “plasmid,” “expression plasmid,” “virus” and “vector” are often used interchangeably.

The term “introduced” in the context of inserting a nucleic acid molecule into a cell, means “transfection”, or “transformation” or “transduction” and includes reference to the incorporation of a nucleic acid molecule into a eukaryotic or prokaryotic cell wherein the nucleic acid molecule may be incorporated into the genome of a cell (e.g., chromosome, plasmid, plastid, or mitochondrial DNA), converted into an autonomous replicon, or transiently expressed (e.g., transfected mRNA). As used herein, the term “engineered,” “recombinant” or “non-natural” refers to an organism, microorganism, cell, nucleic acid molecule, or vector that includes at least one genetic alteration or has been modified by introduction of an exogenous nucleic acid molecule, wherein such alterations or modifications are introduced by genetic engineering (i.e., human intervention). Genetic alterations include, for example, modifications introducing expressible nucleic acid molecules encoding proteins, binding proteins or enzymes, or other nucleic acid molecule additions, deletions, substitutions or other functional disruption of a cell's genetic material. Additional modifications include, for example, non-coding regulatory regions in which the modifications alter expression of a polynucleotide, gene or operon.

As described herein, more than one heterologous nucleic acid molecule can be introduced into a host cell as separate nucleic acid molecules, as a plurality of individually controlled genes, as a polycistronic nucleic acid molecule, as a single nucleic acid molecule encoding a binding protein, or any combination thereof. When two or more heterologous nucleic acid molecules are introduced into a host cell, it is understood that the two or more heterologous nucleic acid molecules can be introduced as a single nucleic acid molecule (e.g., on a single vector), on separate vectors, integrated into the host chromosome at a single site or multiple sites, or any combination thereof. The number of referenced heterologous nucleic acid molecules or protein activities refers to the number of encoding nucleic acid molecules or the number of protein activities, not the number of separate nucleic acid molecules introduced into a host cell.

The term “construct” refers to any polynucleotide that contains a recombinant nucleic acid molecule. A construct may be present in a vector (e.g., a bacterial vector, a viral vector) or may be integrated into a genome. A “vector” is a nucleic acid molecule that is capable of transporting another nucleic acid molecule. Vectors may be, for example, plasmids, cosmids, viruses, a RNA vector or a linear or circular DNA or RNA molecule that may include chromosomal, non-chromosomal, semi-synthetic or synthetic nucleic acid molecules. Vectors of the present disclosure also include transposon systems (e.g., Sleeping Beauty, see, e.g., Geurts et al., Mol. Ther. 8:108, 2003: Mátes et al., Nat. Genet. 41:753 (2009)). Exemplary vectors are those capable of autonomous replication (episomal vector) or expression of nucleic acid molecules to which they are linked (expression vectors).

Viral vectors include retrovirus, adenovirus, parvovirus (e.g., adeno-associated viruses), coronavirus, negative strand RNA viruses such as ortho-myxovirus (e.g., influenza virus), rhabdovirus (e.g., rabies and vesicular stomatitis virus), paramyxovirus (e.g., measles and Sendai), positive strand RNA viruses such as picornavirus and alphavirus, and double-stranded DNA viruses including adenovirus, herpesvirus (e.g., Herpes Simplex virus types 1 and 2, Epstein-Barr virus, cytomegalovirus), and poxvirus (e.g., vaccinia, fowlpox and canarypox). Other viruses include Norwalk virus, togavirus, flavivirus, reoviruses, papovavirus, hepadnavirus, and hepatitis virus, for example. Examples of retroviruses include avian leukosis-sarcoma, mammalian C-type, B-type viruses, D type viruses, HTLV-BLV group, lentivirus, spumavirus (Coffin, J. M., Retroviridae: The viruses and their replication, In Fundamental Virology, Third Edition, B. N. Fields et al., Eds., Lippincott-Raven Publishers, Philadelphia, 1996).

“Retroviruses” are viruses having an RNA genome, which is reverse-transcribed into DNA using a reverse transcriptase enzyme, the reverse-transcribed DNA is then incorporated into the host cell genome. “Gammaretrovirus” refers to a genus of the retroviridae family. Examples of gammaretroviruses include mouse stem cell virus, murine leukemia virus, feline leukemia virus, feline sarcoma virus, and avian reticuloendotheliosis viruses.

“Lentiviral vector,” as used herein, means HIV-based lentiviral vectors for gene delivery, which can be integrative or non-integrative, have relatively large packaging capacity, and can transduce a range of different cell types. Lentiviral vectors are usually generated following transient transfection of three (packaging, envelope and transfer) or more plasmids into producer cells. Like HIV, lentiviral vectors enter the target cell through the interaction of viral surface glycoproteins with receptors on the cell surface. On entry, the viral RNA undergoes reverse transcription, which is mediated by the viral reverse transcriptase complex. The product of reverse transcription is a double-stranded linear viral DNA, which is the substrate for viral integration into the DNA of infected cells. Additional vectors useful for practicing embodiments of the present disclosure are described herein.

As used herein, the term “host” refers to a cell (e.g., T cell) or microorganism targeted for genetic modification with a heterologous nucleic acid molecule to produce a polypeptide of interest (e.g., a binding protein of the present disclosure). In certain embodiments, a host cell may optionally already possess or be modified to include other genetic modifications that confer desired properties related or unrelated to, e.g., biosynthesis of the heterologous protein (e.g., inclusion of a detectable marker; deleted, altered or truncated endogenous TCR; or increased co-stimulatory factor expression).

As used herein, a “hematopoietic progenitor cell” is a cell that can be derived from hematopoietic stem cells or fetal tissue and is capable of further differentiation into mature cells types (e.g., immune system cells). Exemplary hematopoietic progenitor cells include those with a CD24^{Lo} Lin⁻ CD117⁺ phenotype or those found in the thymus (referred to as progenitor thymocytes).

As used herein, an “immune system cell” or “immune cell” means any cell of the immune system that originates from a hematopoietic stem cell in the bone marrow, which gives rise to two major lineages, a myeloid progenitor cell (which give rise to myeloid cells such as monocytes, macrophages, dendritic cells, megakaryocytes and granulocytes) and a lymphoid progenitor cell (which give rise to lymphoid cells such as T cells, B cells, natural killer (NK) cells, and NK-T cells). Exemplary immune system cells include a CD4⁺ T cell, a CD8⁺ T cell, a CD4⁻ CD8⁻ double negative T cell, a γδ T cell, a regulatory T cell, a natural killer cell (e.g., a NK cell or a NK-T cell), and a dendritic cell. Macrophages and dendritic cells may be referred to as “antigen presenting cells” or “APCs,” which are specialized cells that can activate T cells when a major histocompatibility complex (MEW) receptor on the surface of the APC complexed with a peptide interacts with a TCR on the surface of a T cell.

A “T cell” or “T lymphocyte” is an immune system cell that matures in the thymus and produces T cell receptors (TCRs). T cells can be naïve (not exposed to antigen; increased expression of CD62L, CCR7, CD28, CD3, CD127, and CD45RA, and decreased expression of CD45RO as compared to T_{CM}), memory T cells (T_{M}) (antigen-experienced and long-lived), and effector cells (antigen-experienced, cytotoxic). T_{M} can be further divided into subsets of central memory T cells (T_{CM}, increased expression of CD62L, CCR7, CD28, CD127, CD45RO, and CD95, and decreased expression of CD54RA as compared to naïve T cells) and effector memory T cells (T_{EM}, decreased expression of CD62L, CCR7, CD28, CD45RA, and increased expression of CD127 as compared to naïve T cells or T_{CM}).

Effector T cells (T_{E}) refers to antigen-experienced CD8⁺ cytotoxic T lymphocytes that has decreased expression of CD62L, CCR7, CD28, and are positive for granzyme and perforin as compared to T_{CM}. Helper T cells (T_{H}) are CD4⁺ cells that influence the activity of other immune cells by releasing cytokines. CD4⁺ T cells can activate and suppress an adaptive immune response, and which of those two functions is induced will depend on presence of other cells and signals. T cells can be collected using known techniques, and the various subpopulations or combinations thereof can be enriched or depleted by known techniques, such as by affinity binding to antibodies, flow cytometry, or immunomagnetic selection. Other exemplary T cells include regulatory T cells, such as CD4⁺ CD25⁺ (Foxp3⁺) regulatory T cells, stem cell memory T cells, and Treg17 cells, as well as Tr1, Th3, CD8⁺CD28⁻, and Qa-1 restricted T cells.

“Cells of T cell lineage” refer to cells that show at least one phenotypic characteristic of a T cell, or a precursor or progenitor thereof that distinguishes the cells from other lymphoid cells, and cells of the erythroid or myeloid lineages. Such phenotypic characteristics can include expression of one or more proteins specific for T cells (e.g., CD3⁺, CD4⁺, CD8⁺), or a physiological, morphological, functional, or immunological feature specific for a T cell. For example, cells of the T cell lineage may be progenitor or precursor cells committed to the T cell lineage; CD25⁺ immature and inactivated T cells; cells that have undergone CD4 or CD8 linage commitment; thymocyte progenitor cells that are CD4⁺CD8⁺ double positive; single positive CD4⁺ or CD8⁺; TCRαβ or TCR γδ; or mature and functional or activated T cells.

As used herein, “hyperproliferative disease” refers to excessive growth or proliferation as compared to a normal or undiseased cell. Exemplary hyperproliferative disorders include tumors, cancers, neoplastic tissue, carcinoma, sarcoma, malignant cells, pre-malignant cells, as well as non-neoplastic or non-malignant hyperproliferative disorders (e.g., adenoma, fibroma, lipoma, leiomyoma, hemangioma, fibrosis, restenosis, as well as autoimmune diseases such as rheumatoid arthritis, osteoarthritis, psoriasis, inflammatory bowel disease, or the like). Certain diseases that involve abnormal or excessive growth that occurs more slowly than in the context of a hyperproliferative disease can be referred to as “proliferative diseases”, and include certain tumors, cancers, neoplastic tissue, carcinoma, sarcoma, malignant cells, pre-malignant cells, as well as non-neoplastic or non-malignant disorders “Treat” or “treatment” or “ameliorate” refers to medical management of a disease, disorder, or condition of a subject (e.g., a human or non-human mammal, such as a primate, horse, cat, dog, goat, mouse, or rat). In general, an appropriate dose or treatment regimen comprising a host cell expressing a binding protein of the present disclosure, and optionally an adjuvant, is administered in an amount sufficient to elicit a therapeutic or prophylactic benefit. Therapeutic or prophylactic/preventive benefit includes improved clinical outcome; lessening or alleviation of symptoms associated with a disease; decreased occurrence of symptoms; improved quality of life; longer disease-free status; diminishment of extent of disease, stabilization of disease state; delay of disease progression; remission; survival; prolonged survival; or any combination thereof.

As used herein, “statistically significant” refers to a p value of 0.050 or less when calculated using the Students t-test and indicates that it is unlikely that a particular event or result being measured has arisen by chance.

As used herein, the term “adoptive immune therapy” or “adoptive immunotherapy” refers to administration of naturally occurring or genetically engineered, disease antigen-specific immune cells (e.g., T cells). Adoptive cellular immunotherapy may be autologous (immune cells are from the recipient), allogeneic (immune cells are from a donor of the same species) or syngeneic (immune cells are from a donor genetically identical to the recipient).

### Binding Proteins, Polynucleotides, and Modified Immune Cells

In certain aspects, the present disclosure provides binding proteins that specifically bind to an antigen expressed by or associated with a disease or disorder. A “binding protein” refers herein to a protein or polypeptide that possesses the ability to specifically and non-covalently associate, unite, or combine with a target (e.g., a proliferative disease- or hyperproliferative disease-associated antigen). In certain embodiments, a binding protein of the present disclosure comprises a binding domain that specifically binds to an antigen that is expressed by a diseased cell or is otherwise associated with a disease or disorder. A “binding domain” (also referred to as a “binding region” or “binding moiety”), as used herein, refers to a molecule or portion thereof (e.g., peptide, oligopeptide, polypeptide, protein (e.g., a binding protein)) that possesses the ability to specifically and non-covalently associate, unite, or combine with a target (e.g., a proliferative or hyperproliferative disease-associated antigen). A binding domain includes any naturally occurring, synthetic, semi-synthetic, or recombinantly produced binding partner for a biological molecule, a molecular complex (i.e., complex comprising two or more biological molecules), or other target of interest. Exemplary binding domains include single chain immunoglobulin variable regions (e.g., scTCR, scFv, Fab, TCR variable regions), Fabs, receptor ectodomains, ligands (e.g., cytokines, chemokines), or synthetic polypeptides selected for their specific ability to bind to a biological molecule, a molecular complex or other target of interest.

“Antigen” or “Ag” as used herein refers to an immunogenic molecule that provokes an immune response. This immune response may involve antibody production, activation of specific immunologically-competent cells (e.g., T cells), or both. An antigen (immunogenic molecule) may be, for example, a peptide, glycopeptide, polypeptide, glycopolypeptide, polynucleotide, polysaccharide, lipid or the like. It is readily apparent that an antigen can be synthesized, produced recombinantly, or derived from a biological sample. Exemplary biological samples that can contain one or more antigens include tissue samples, tumor samples, cells, biological fluids, or combinations thereof. Antigens can be produced by cells that have been modified or genetically engineered to express an antigen.

The term “epitope” or “antigenic epitope” includes any molecule, structure, amino acid sequence or protein determinant that is recognized and specifically bound by a cognate binding molecule, such as an immunoglobulin, T cell receptor (TCR), chimeric antigen receptor, or other binding molecule, domain or protein. Epitopic determinants generally contain chemically active surface groupings of molecules, such as amino acids or sugar side chains, and can have specific three dimensional structural characteristics, as well as specific charge characteristics.

The terms “complementarity determining region,” and “CDR,” are synonymous with “hypervariable region” or “HVR,” and are known in the art to refer to sequences of amino acids within TCR or antibody variable regions, which confer antigen specificity and/or binding affinity and are separated from one another by framework sequences or regions. In general, there are three CDRs in each variable region (e.g., in the case of a TCR, αCDR1, αCDR2, αCDR3, βCDR1, βCDR2, and βCDR3; in the case of an antibody, HCDR1, HCDR2, HCDR3, LCDR1, LCDR2, and LCDR3). In the case of TCRs, CDR3 is thought to be the main CDR responsible for recognizing processed antigen. CDR1 and CDR2 mainly interact with the MHC. As used herein, a “variant” of a CDR refers to a functional variant of a CDR sequence (i.e., capable of binding the target antigen with an avidity or affinity similar to the unmodified CDR, such as within about 30%, about 25%, about 20%, about 15%, about 10%, about 5% of the avidity or affinity of the unmodified CDR, or has the same or improved avidity or affinity as the unmodified CDR, such as about 5%, about 10%, about 15%, about 20%, about 25%, or about 30% improved). An exemplary variant CDR may have up to 1-3 amino acid substitutions, deletions, or combinations thereof, provided it is functional (binding) variant CDR.

As used herein, “specifically binds” or “specific for” refers to an association or union of a binding protein (e.g., a T cell receptor or a chimeric antigen receptor) or a binding domain (or binding protein thereof) to a target molecule (e.g., an antigen that is associated with a proliferative disease such as a cancer) with an affinity or Kₐ (i.e., an equilibrium association constant of a particular binding interaction with units of 1/M) equal to or greater than 10⁵M⁻¹ (which equals the ratio of the on-rate [Kₒₙ] to the off rate [K_{off}] for this association reaction), while not significantly associating or uniting with any other molecules or components in a sample. Binding proteins or binding domains (or binding proteins thereof) may be classified as “high-affinity” binding proteins or binding domains (or binding proteins thereof) or as “low-affinity” binding proteins or binding domains (or binding proteins thereof). “High-affinity” binding proteins or binding domains refer to those binding proteins or binding domains having a Kₐ of at least 10⁷M⁻¹, at least 10⁸ M⁻¹, at least 10⁹ M¹, at least 10¹⁰ M⁻¹, at least 10¹¹ M⁻¹, at least 10¹²M⁻¹, or at least 10¹³M⁻¹. “Low-affinity” binding proteins or binding domains refer to those binding proteins or binding domains having a Kₐ of up to 10⁷ M⁻¹, up to 10⁶ M⁻¹, up to 10⁵ M⁻¹. Alternatively, affinity may be defined as an equilibrium dissociation constant (Kd) of a particular binding interaction with units of M (e.g., 10⁻⁵ M to 10⁻¹³M).

In certain embodiments, a receptor or binding domain may have “enhanced affinity,” which refers to selected or engineered receptors or binding domains with stronger binding to a target antigen than a wild type (or parent) binding domain. For example, enhanced affinity may be due to a Kₐ (equilibrium association constant) for the target antigen that is higher than the wild type binding domain, due to a K_{d} (dissociation constant) for the target antigen that is less than that of the wild type binding domain, due to an off-rate (k_{off}) for the target antigen that is less than that of the wild type binding domain, or a combination thereof. In certain embodiments, binding proteins may be codon optimized to enhance expression in a particular host cell, such as T cells (e.g., Scholten et al., Clin. Immunol. 119:135, 2006).

A variety of assays are known for identifying binding domains of the present disclosure that specifically bind a particular target, as well as determining binding domain or binding protein affinities, such as Western blot, ELISA, analytical ultracentrifugation, spectroscopy and surface plasmon resonance (Biacore®) analysis (see, e.g., Scatchard et al., Ann. N.Y. Acad. Sci. 51:660, 1949; Wilson, Science 295:2103, 2002; Wolff et al., Cancer Res. 53:2560, 1993; and U.S. Pat. Nos. 5,283,173, 5,468,614, or the equivalent). Assays for assessing affinity or apparent affinity or relative affinity are also known. In certain examples, apparent affinity for a binding protein is measured by assessing binding to various concentrations of tetramers, for example, by flow cytometry using labeled tetramers. In some examples, apparent K_{D} of a binding protein is measured using 2-fold dilutions of labeled tetramers at a range of concentrations, followed by determination of binding curves by non-linear regression, apparent K_{D} being determined as the concentration of ligand that yielded half-maximal binding.

In certain embodiments, the binding domain is a scFv comprising heavy chain and light chain variable regions connected by short linker peptide. Any scFv of the present disclosure may be engineered so that the C-terminal end of V_{L} domain is linked by a short peptide sequence to the N-terminal end of the V_{H} domain, or vice versa (i.e., (N)V_{L}(C)-linker-(N)V_{H}(C) or (N)V_{H}(C)-linker-(N)V_{L}(C)).

In some embodiments, the binding protein specifically binds to a tumor-associated antigen selected from ROR1, EGFR, EGFRvIII, EGP-2, EGP-40, GD2, GD3, HPV E6, HPV E7, Her2, L1-CAM, Lewis A, Lewis Y, MUC1, MUC16, PSCA, PSMA, CD19, CD20, CD22, CD56, CD23, CD24, CD30, CD33, CD37, CD44v7/8, CD38, CD56, CD123, CA125, c-MET, FcRH5, WT1, folate receptor α, VEGF-α, VEGFR1, VEGFR2, IL-13Rα2, IL-11Rα, MAGE-A1, PSA, ephrin A2, ephrin B2, an NKG2D, NY-ESO-1, TAG-72, mesothelin, NY-ESO, 5T4, BCMA, FAP, Carbonic anhydrase 9, ERBB2, BRAFV600E, or CEA.

In some embodiments, the tumor associated antigen is ROR1. In particular embodiments, the binding protein comprises a binding domain derived from antibody R12, antibody 2A2, antibody R11, antibody UC-961, antibody D10, or antibody H10. Incorporated herein by reference are all the ROR1 antibodies and related protein and nucleic acid constructs and related sequences disclosed in: PCT Publication Nos. WO 2014/031687, WO 2014/031174, and WO 2016/094873; U.S. Pat. Nos. 9,316,646, 9,242,014, 9,217,040, 9,228,023, 9,150,647, 9,266,952, and 8,212,009; and in U.S. Published Patent Application No. US 2013/025164.

In some embodiments, the antigen-specific receptor binding domain is derived from antibody 2A2, antibody R12, antibody R11, antibody Y31, antibody UC-961, antibody D10, or antibody H10 and has a VH, or (i.e., and/or) a VL having at least about 80%, 85%, 90%, 91%, 92%, 93%, 94%, 95%, 96%, 96%, 97%, 98%, 99%, or more amino acid sequence identity to that of the antibody variable regions or scFv thereof from antibody R12, antibody 2A2, antibody R12, antibody R11, antibody Y31, antibody UC-961, antibody D10, or antibody H10. CDR, VH, and VL sequences from these exemplary antibodies, and scFvs derived therefrom, are provided in SEQ ID NOs:1-45, and any of these sequences (or functional variants of) may be present in a ROR1-specific binding domain of the present disclosure.

In certain embodiments, the antigen-specific receptor binding domain comprises HCDR1, HCDR2, HCDR3, LCDR1, LCDR2, and LCDR3 amino acid sequences as set forth in SEQ ID NOs:1-6, respectively. In certain embodiments, the antigen-specific receptor binding domain comprises a VH comprising or consisting of an amino acid sequence having at least 80%, 85%, 90%, 91%, 92%, 93%, 94%, 95%, 96%, 96%, 97%, 98%, 99%, or more amino acid sequence identity to SEQ ID NO:7, and a VL comprising or consisting of an amino acid sequence having at least 80%, 85%, 90%, 91%, 92%, 93%, 94%, 95%, 96%, 96%, 97%, 98%, 99%, or more amino acid sequence identity to SEQ ID NO:8. In certain embodiments, the antigen-specific receptor binding domain comprises a scFv comprising or consisting of an amino acid sequence having at least 80%, 85%, 90%, 91%, 92%, 93%, 94%, 95%, 96%, 96%, 97%, 98%, 99%, or more amino acid sequence identity to SEQ ID NO:9.

In certain embodiments, the binding protein comprises a transmembrane component or transmembrane domain disposed between an extracellular component comprising the binding domain and an intracellular component, which can comprise an effector domain. As used herein, an “effector domain” is an intracellular portion or domain of a binding protein or receptor that can directly or indirectly promote a biological or physiological response in a cell when receiving an appropriate signal. In certain embodiments, an effector domain is from a protein or portion thereof or protein complex that receives a signal when bound, or when the protein or portion thereof or protein complex binds directly to a target molecule and triggers a signal from the effector domain.

An effector domain may directly promote a cellular response when it contains one or more signaling domains or motifs, such as an Intracellular Tyrosine-based Activation Motif (ITAM), as found in costimulatory molecules. Without wishing to be bound by theory, it is believed the ITAMs are important for T cell activation following ligand engagement by a T cell receptor or by a binding protein comprising a T cell effector domain. In certain embodiments, the intracellular component comprises an ITAM. Exemplary effector domains include those from CD27, CD28, 4-1BB (CD137), OX40 (CD134), CD3ε, CD3δ, CD3ζ, CD25, CD27, CD28, CD79A, CD79B, CARD11, DAP10, FcRα, FcRβ, FcRγ, Fyn, HVEM, ICOS, Lck, LAG3, LAT, LRP, NKG2D, NOTCH1, NOTCH2, NOTCH3, NOTCH4, Wnt, ROR2, Ryk, SLAMF1, Slp76, pTa, TCRα, TCRβ, TRIM, Zap70, PTCH2, or any combination thereof. In certain embodiments, an effector domain comprises a lymphocyte receptor signaling domain CD3).

In further embodiments, the intracellular component of the binding protein comprises a costimulatory domain or portion thereof selected from CD27, CD28, 4-1BB (CD137), OX40 (CD134), ICOS (CD278), CD27, CD2, CD5, ICAM-1 (CD54), LFA-1 (CD11a/CD18), GITR, CD30, CD40, BAFF-R, HVEM, LIGHT, NKG2C, SLAMF7, NKp80, CD160, B7-H3 CD94, DAP12, a ligand that specifically binds with CD83, or a functional variant thereof, or any combination thereof. In certain embodiments, the intracellular component comprises a CD28 costimulatory domain or portion thereof (which may optionally include a LL→GG mutation at positions 186-187 of the native CD28 protein; see Nguyen et al., Blood 102:4320, 2003)), a 4-1BB costimulatory domain or portion thereof, or both.

In certain embodiments, an effector domain comprises CD3 or a functional portion thereof. In further embodiments, an effector domain comprises a portion or a domain from CD27. In some embodiments, an effector domain comprises a portion or a domain from CD28. In some embodiments, an effector domain comprises a portion or a domain from 4-1BB. In some embodiments, an effector domain comprises a portion or a domain from OX40. In some embodiments, an effector domain comprises a portion or a domain from 4-1BB and a portion or a domain from CD3.

In some embodiments, an extracellular component and an intracellular component of a binding protein of the present disclosure are connected by a transmembrane component or domain. A “transmembrane component” or “transmembrane domain”, as used herein, is a portion of a transmembrane protein that can insert into or span a cell membrane. Transmembrane components or domains have a three-dimensional structure that is thermodynamically stable in a cell membrane and generally range in length from about 15 amino acids to about 30 amino acids. The structure of a transmembrane component or domain may comprise an alpha helix, a beta barrel, a beta sheet, a beta helix, or any combination thereof. In certain embodiments, a transmembrane component or domain comprises or is derived from a known transmembrane protein (e.g., a CD4 transmembrane domain, a CD8 transmembrane domain, a CD27 transmembrane domain, a CD28 transmembrane domain, or any combination thereof). In particular embodiments, a transmembrane component of a binding protein is derived from CD28 and an intracellular component effector domain comprises a 4-1BB signaling domain and a CD3t domain.

In certain embodiments, the extracellular component of the binding protein further comprises a linker (also referred to herein as a “spacer”) disposed between the binding domain and the transmembrane domain. As used herein when referring to a component of a binding protein that connects the binding and transmembrane domains, a “linker” may be an amino acid sequence having from about two amino acids to about 500 amino acids, which can provide flexibility and room for conformational movement between two regions, domains, motifs, fragments, or modules connected by the linker. For example, a linker of the present disclosure can position the binding domain away from the surface of a host cell expressing the binding protein to enable proper contact between the host cell and a target cell, antigen binding, and activation (Patel et al., Gene Therapy 6: 412-419, 1999). Linker length may be varied to maximize antigen recognition based on the selected target molecule, selected binding epitope, or antigen binding domain seize and affinity (see, e.g., Guest et al., J. Immunother. 28:203-11, 2005; PCT Publication No. WO 2014/031687). Exemplary linkers include those having a glycine-serine amino acid chain having from one to about ten repeats of GlyₓSer_{y}, wherein x and y are each independently an integer from 0 to 10, provided that x and y are not both 0 (e.g., (Gly₄Ser)₂, (Gly₃Ser)₂, Gly₂Ser, or a combination thereof, such as ((Gly₃Ser)₂Gly₂Ser).

Linkers of the present disclosure also include immunoglobulin constant regions (i.e., CH1, CH2, CH3, or CL, of any isotype) and portions thereof. In certain embodiments, a linker comprises a CH3 domain, a CH2 domain, or both. In certain embodiments, a linker comprises a CH2 domain and a CH3 domain. In further embodiments, the CH2 domain and the CH3 domain are each a same isotype. In particular embodiments, the CH2 domain and the CH3 domain are an IgG4 or IgG1 isotype. In other embodiments, the CH2 domain and the CH3 domain are each a different isotype. In specific embodiments, the CH2 comprises a N297Q mutation. Without wishing to be bound by theory, it is believed that CH2 domains with N297Q mutation do not bind FcγR (see, e.g., Sazinsky et al., PNAS 105(51):20167 (2008)). In certain embodiments, the linker comprises a human immunoglobulin constant region or portion thereof. An exemplary IgG4 Fc polypeptide sequence is provided in SEQ ID NO:49.

In any of the embodiments described herein, a linker may comprise a hinge region or portion thereof. Hinge regions are flexible amino acid polymers of variable length and sequence (typically rich in proline and cysteine amino acids) and connect larger and less-flexible regions of immunoglobulin proteins. For example, hinge regions connect the heavy chain constant and Fab regions of antibodies and connect the constant and transmembrane regions of TCRs.

Linkers comprising modified immunoglobulin constant or hinge regions, or portions, thereof, are also contemplated, wherein the modification (e.g., substitution, insertion, deletion) does not substantially affect one or more functional characteristic of interest (e.g. length, flexibility, solubility) of the linker. In some embodiments, the linker comprises a constant region, hinge region, modified constant region, or modified hinge region, that is, or is derived from, an IgG isotype, such as, for example, an IgG1, IgG2, IgG3, or IgG4 isotype.

In certain embodiments, one or more of the extracellular component, the binding domain, the linker, the transmembrane domain, the intracellular component, or the costimulatory domain comprises a junction amino acid. “Junction amino acids” or “junction amino acid residues” refer to one or more (e.g., about 2-20) amino acid residues between two adjacent domains, motifs, regions, modules, or fragments of a protein, such as between a binding domain and an adjacent linker, between a transmembrane domain and an adjacent extracellular or intracellular domain, or on one or both ends of a linker that links two domains, motifs, regions, modules, or fragments (e.g., between a linker and an adjacent binding domain or between a linker and an adjacent hinge). Junction amino acids may result from the construct design of a binding protein (e.g., amino acid residues resulting from the use of a restriction enzyme site or self-cleaving peptide sequences during the construction of a polynucleotide encoding a binding protein). For example, a transmembrane domain of a binding protein may have one or more junction amino acids at the amino-terminal end, carboxy-terminal end, or both.

In some embodiments, a binding protein of the present disclosure may further comprise a protein tag (also called a peptide tag or tag peptide herein). Protein tags are unique peptide sequences that are affixed or genetically fused to, or are a part of, a protein of interest and can be recognized or bound by, for example, a heterologous or non-endogenous cognate binding molecule or a substrate (e.g., receptor, ligand, antibody, carbohydrate, or metal matrix). Protein tags are useful for detecting, identifying, isolating, tracking, purifying, enriching for, targeting, or biologically or chemically modifying tagged proteins of interest, particularly when a tagged protein is part of a heterogenous population of cells (e.g., a biological sample like peripheral blood). In the provided binding proteins, the ability of the tag(s) to be specifically bound by the cognate binding molecules is distinct from, or in addition to, the ability of the binding domain(s) to specifically bind the hyperproliferative disease-associated antigen. In certain embodiments, the protein tag is a Myc tag, His tag, Flag tag, Xpress tag, Avi tag, Calmodulin tag, Polyglutamate tag, HA tag, Nus tag, S tag, X tag, SBP tag, Softag, V5 tag, CBP, GST, MBP, GFP, Thioredoxin tag, Strep-Tag (e.g., Strep-Tag® or Strep-Tag II®), or any combination thereof.

In any of the embodiments described herein, a binding protein can be or can comprise a TCR or a CAR, or both. “T cell receptor” (TCR) refers to an immunoglobulin superfamily member (having a variable binding domain, a constant domain, a transmembrane region, and a short cytoplasmic tail; see, e.g., Janeway et al., Immunobiology: The Immune System in Health and Disease, 3^{rd} Ed., Current Biology Publications, p. 4:33, 1997) capable of specifically binding to an antigen peptide bound to a MHC receptor. A TCR can be found on the surface of a cell or in soluble form and generally is comprised of a heterodimer having α and δ chains (also known as TCa and TCRβ, respectively), or γ and δ chains (also known as TCRγ and TCRδ, respectively). Like other immunoglobulins (e.g., antibodies), the extracellular portion of TCR chains (e.g., α-chain, β-chain) contain two immunoglobulin domains, a variable domain (e.g., α-chain variable domain or V_{α}, β-chain variable domain or V_{β}; typically amino acids 1 to 116 based on Kabat numbering (Kabat et al., “Sequences of Proteins of Immunological Interest, US Dept. Health and Human Services, Public Health Service National Institutes of Health, 1991, 5^{th} ed.) at the N-terminus, and one constant domain (e.g., α-chain constant domain or C_{α}, typically amino acids 117 to 259 based on Kabat, β-chain constant domain or C_{β}, typically amino acids 117 to 295 based on Kabat) adjacent to the cell membrane. Also, like antibodies, the variable domains contain complementary determining regions (CDRs) separated by framework regions (FRs) (see, e.g., Jores et al., Proc. Nat'l Acad. Sci. U.S.A. 87:9138, 1990; Chothia et al., EMBO J. 7:3745, 1988; see also Lefranc et al., Dev. Comp. Immunol. 27:55, 2003). In certain embodiments, a TCR is found on the surface of T cells (or T lymphocytes) and associates with the CD3 complex. The source of a TCR as used in the present disclosure may be from various animal species, such as a human, mouse, rat, rabbit or other mammal. Methods for producing engineered TCRs are described in, for example, Bowerman et al., Mol. Immunol., 46(15):3000 (2009), the techniques of which are herein incorporated by reference.

“CD3” is a multi-protein complex of six chains (see, Abbas and Lichtman, 2003; Janeway et al., p. 172 and 178, 1999) that is associated with antigen signaling in T cells. In mammals, the complex comprises a CD3γ chain, a CD3δ chain, two CD3ε chains, and a homodimer of CD3ζ chains. The CD3γ, CD3β, and CD3ε chains are related cell surface proteins of the immunoglobulin superfamily containing a single immunoglobulin domain. The transmembrane regions of the CD3γ, CD3β, and CD3ε chains are negatively charged, which is believed to allow these chains to associate with positively charged regions of T cell receptor chains. The intracellular tails of the CD3γ, CD3β, and CD3ε chains each contain a single conserved motif known as an immunoreceptor tyrosine based activation motif or ITAM, whereas each CD3 chain has three. Without wishing to be bound by theory, it is believed that the ITAMs are important for the signaling capacity of a TCR complex. CD3 as used in the present disclosure may be from various animal species, including human, mouse, rat, or other mammals.

As used herein, “TCR complex” refers to a complex formed by the association of CD3 with TCR. For example, a TCR complex can be composed of a CD3γ chain, a CD3β chain, two CD3ε chains, a homodimer of CD3ζ chains, a TCRα chain, and a TCRβ chain. Alternatively, a TCR complex can be composed of a CD3γ chain, a CD3β chain, two CD3ε chains, a homodimer of CD3ζ chains, a TCRγ chain, and a TCRβ chain.

A “component of a TCR complex”, as used herein, refers to a TCR chain (i.e., TCRα, TCRβ, TCRγ or TCRδ), a CD3 chain (i.e., CD3γ, CD3δ, CD3ε or CD3ζ), or a complex formed by two or more TCR chains or CD3 chains (e.g., a complex of TCRα and TCRβ, a complex of TCRγ and TCRδ, a complex of CD3ε and CD3δ, a complex of CD3γ and CD3ε, or a sub-TCR complex of TCRα, TCRβ, CD3γ, CD3δ, and two CD3ε chains).

“Major histocompatibility complex molecules” (MHC molecules) refer to glycoproteins that deliver peptide antigens to a cell surface. MHC class I molecules are heterodimers consisting of a membrane spanning a chain (with three α domains) and a non-covalently associated β2 microglobulin. MHC class II molecules are composed of two transmembrane glycoproteins, α and β, both of which span the membrane. Each chain has two domains. MHC class I molecules deliver peptides originating in the cytosol to the cell surface, where a peptide:MHC complex is recognized by CD8⁺ T cells. MHC class II molecules deliver peptides originating in the vesicular system to the cell surface, where they are recognized by CD4⁺ T cells. An MHC molecule may be from various animal species, including human, mouse, rat, cat, dog, goat, horse, or other mammals.

“Chimeric antigen receptor” (CAR) refers to a binding protein of the present disclosure engineered to contain two or more naturally-occurring amino acid sequences linked together in a way that does not occur naturally or does not occur naturally in a host cell, which binding protein can function as a receptor when present on a surface of a cell. CARs of the present disclosure include an extracellular portion comprising an antigen-binding domain (e.g., obtained or derived from an immunoglobulin or immunoglobulin-like molecule, such as an scFv derived from an antibody or a scTCR derived from a TCR specific for a cancer antigen, or an antigen-binding domain derived or obtained from a killer immunoreceptor from an NK cell) linked to a transmembrane domain and one or more intracellular signaling domains (optionally containing co-stimulatory domain(s)) (see, e.g., Sadelain et al., Cancer Discov., 3(4):388 (2013); see also Harris and Kranz, Trends Pharmacol. Sci., 37(3):220 (2016); Stone et al., Cancer Immunol. Immunother., 63(11):1163 (2014)).

Methods of making binding proteins, including CARs, are known in the art and are described, for example, in U.S. Pat. Nos. 6,410,319; 7,446,191; U.S. Patent Publication No. 2010/065818; U.S. Pat. No. 8,822,647; PCT Publication No. WO 2014/031687; U.S. Pat. No. 7,514,537; and Brentjens et al., 2007, Clin. Cancer Res. 13:5426, the techniques of which are herein incorporated by reference.

In certain embodiments, the antigen-binding fragment of the TCR comprises a single chain TCR (scTCR), which comprises both the TCR Vα and TCR Vβ domains, but only a single TCR constant domain (Cα or Cβ). In certain embodiments, the antigen-binding fragment of the TCR or CAR is chimeric (e.g., comprises amino acid residues or motifs from more than one donor or species), humanized (e.g., comprises residues from a non-human organism that are altered or substituted so as to reduce the risk of immunogenicity in a human), or human.

In certain embodiments, a binding protein comprises a TCR-CAR, which generally comprises at least a soluble antigen-binding portion of a TCR fused to a CAR intracellular signaling domain(s) (see, e.g., Walseng et al., Scientific Reports 7:10713 (2017), the TCR-CAR constructs of which are hereby incorporated by reference in their entirety).

Methods useful for isolating and purifying recombinantly produced soluble binding proteins, by way of example, may include obtaining supernatants from suitable host cell/vector systems that secrete the recombinant soluble binding protein into culture media and then concentrating the media using a commercially available filter. Following concentration, the concentrate may be applied to a single suitable purification matrix or to a series of suitable matrices, such as an affinity matrix or an ion exchange resin. One or more reverse phase HPLC steps may be employed to further purify a recombinant polypeptide. These purification methods may also be employed when isolating an immunogen from its natural environment. Methods for large scale production of one or more of the isolated/recombinant soluble binding protein described herein include batch cell culture, which is monitored and controlled to maintain appropriate culture conditions. Purification of the soluble binding protein may be performed according to methods described herein and known in the art and that comport with laws and guidelines of domestic and foreign regulatory agencies.

Binding proteins as described herein may be functionally characterized according to any of a large number of art-accepted methodologies for assaying host cell (e.g., T cell) activity, including, for example, determination of host cell (e.g., T cell) binding, activation or induction and also including determination of host cell responses that are antigen-specific. Examples include determination of T cell proliferation, T cell cytokine release, antigen-specific T cell stimulation, MHC restricted T cell stimulation, CTL activity (e.g., by detecting ⁵¹Cr or Europium release from pre-loaded target cells), changes in T cell phenotypic marker expression, and other measures of T-cell functions. Procedures for performing these and similar assays are may be found, for example, in Lefkovits (Immunology Methods Manual: The Comprehensive Sourcebook of Techniques, 1998). See, also, Current Protocols in Immunology; Weir, Handbook of Experimental Immunology, Blackwell Scientific, Boston, Mass. (1986); Mishell and Shigii (eds.) Selected Methods in Cellular Immunology, Freeman Publishing, San Francisco, C A (1979); Green and Reed, Science 281:1309 (1998) and references cited therein.

Levels of cytokines may be determined according to methods described herein and practiced in the art, including for example, ELISA, ELISPOT, intracellular cytokine staining, and flow cytometry and combinations thereof (e.g., intracellular cytokine staining and flow cytometry). Immune cell proliferation and clonal expansion resulting from an antigen-specific elicitation or stimulation of an immune response may be determined by isolating lymphocytes, such as circulating lymphocytes in samples of peripheral blood cells or cells from lymph nodes, stimulating the cells with antigen, and measuring cytokine production, cell proliferation and/or cell viability, such as by incorporation of tritiated thymidine or non-radioactive assays, such as MTT assays and the like. The effect of an immunogen described herein on the balance between a Th1 immune response and a Th2 immune response may be examined, for example, by determining levels of Th1 cytokines, such as IFN-γ, IL-12, IL-2, and TNF-β, and Type 2 cytokines, such as IL-4, IL-5, IL-9, IL-10, and IL-13.

In another aspect, nucleic acid molecules are provided that encode any one or more of the binding proteins described herein. A polynucleotide encoding a desired binding protein can be obtained or produced using recombinant methods known in the art using standard techniques, such as screening libraries from cells expressing a desired sequence or a portion thereof, by deriving a sequence from a vector known to include the same, or by isolating a sequence or a portion thereof directly from cells or tissues containing the same. Alternatively, a sequence of interest can be produced synthetically. Such nucleic acid molecules can be inserted into an appropriate vector (e.g., viral vector or non-viral plasmid vector) for introduction into a host cell of interest (e.g., an immune cell, such as a T cell).

Markers may be used to identify or monitor expression of a heterologous polynucleotide by a host cell transduced with the same, or to detect cells expressing a binding protein of interest. In certain embodiments, the polynucleotide encoding the marker is located 3′ to the polynucleotide encoding the intracellular component of the binding protein, or is located 5′ to the polynucleotide encoding the extracellular component. Exemplary markers include green fluorescent protein, an extracellular domain of human CD2, a truncated human EGFR (huEGFRt; see Wang et al., Blood 118:1255 (2011)), a truncated human CD19 (huCD19t), a truncated human CD34 (huCD34t), or a truncated human NGFR (huNGFRt. In certain embodiments, the encoded marker comprises EGFRt, CD19t, CD34t, or NGFRt.

In any of the embodiments described herein, a binding-protein-encoding polynucleotide can further comprise a polynucleotide that encodes a self-cleaving polypeptide, wherein the polynucleotide encoding the self-cleaving polypeptide is located between the polynucleotide encoding the intracellular component and the polynucleotide encoding the marker. When the polynucleotide is expressed by a host cell comprising the same, the binding protein and the marker are expressed as separate molecules at the host cell surface.

In certain embodiments, a self-cleaving polypeptide comprises a 2A peptide from porcine teschovirus-1 (P2A), Thosea asigna virus (T2A), equine rhinitis A virus (E2A), or foot-and-mouth disease virus (F2A)). Further exemplary nucleic acid and amino acid sequences of 2A peptides are set forth in, for example, Kim et al. (PLOS One 6:e18556, 2011, which 2A nucleic acid and amino acid sequences are incorporated herein by reference in their entirety; see also SEQ ID NOs:50-57).

In any of the embodiments described herein, a polynucleotide of the present disclosure may be codon optimized for a host cell containing the polynucleotide (see, e.g., Scholten et al., Clin. Immunol. 119:135-145 (2006)).

In further aspects, expression constructs are provided, wherein the expression constructs comprise a polynucleotide of the present disclosure operably linked to an expression control sequence (e.g., a promoter). In certain embodiments, the expression construct is comprised in a vector. An exemplary vector may comprise a polynucleotide capable of transporting another polynucleotide to which it has been linked, or which is capable of replication in a host organism. Some examples of vectors include plasmids, viral vectors, cosmids, and others. Some vectors may be capable of autonomous replication in a host cell into which they are introduced (e.g. bacterial vectors having a bacterial origin of replication and episomal mammalian vectors), whereas other vectors may be integrated into the genome of a host cell or promote integration of the polynucleotide insert upon introduction into the host cell and thereby replicate along with the host genome (e.g., lentiviral vector, retroviral vector). Additionally, some vectors are capable of directing the expression of genes to which they are operatively linked (these vectors may be referred to as “expression vectors”). According to related embodiments, it is further understood that, if one or more agents (e.g., polynucleotides encoding fusion proteins as described herein) are co-administered to a subject, that each agent may reside in separate or the same vectors, and multiple vectors (each containing a different agent or the same agent) may be introduced to a cell or cell population or administered to a subject.

In certain embodiments, polynucleotides of the present disclosure may be operatively linked to certain elements of a vector. For example, polynucleotide sequences that are needed to effect the expression and processing of coding sequences to which they are ligated may be operatively linked. Expression control sequences may include appropriate transcription initiation, termination, promoter and enhancer sequences; efficient RNA processing signals such as splicing and polyadenylation signals; sequences that stabilize cytoplasmic mRNA; sequences that enhance translation efficiency (i.e., Kozak consensus sequences); sequences that enhance protein stability; and possibly sequences that enhance protein secretion. Expression control sequences may be operatively linked if they are contiguous with the gene of interest and expression control sequences that act in trans or at a distance to control the gene of interest.

In certain embodiments, the vector comprises a plasmid vector or a viral vector (e.g., a vector selected from lentiviral vector or a γ-retroviral vector). In certain embodiments, the viral vector can be a gammaretrovirus, e.g., Moloney murine leukemia virus (MLV)-derived vectors. In other embodiments, the viral vector can be a more complex retrovirus-derived vector, e.g., a lentivirus-derived vector. HIV-1-derived vectors belong to this category. Other examples include lentivirus vectors derived from HIV-2, FIV, equine infectious anemia virus, SIV, and Maedi-Visna virus (ovine lentivirus). Methods of using retroviral and lentiviral viral vectors and packaging cells for transducing mammalian host cells with viral particles containing CAR transgenes are known in the art and have been previous described, for example, in: U.S. Pat. No. 8,119,772; Walchli et al., PLoS One 6:327930, 2011; Zhao et al., J. Immunol. 174:4415, 2005; Engels et al., Hum. Gene Ther. 14:1155, 2003; Frecha et al., Mol. Ther. 18:1748, 2010; and Verhoeyen et al., Methods Mol. Biol. 506:97, 2009. Retroviral and lentiviral vector constructs and expression systems are also commercially available. Other viral vectors also can be used for polynucleotide delivery including DNA viral vectors, including, for example adenovirus-based vectors and adeno-associated virus (AAV)-based vectors; vectors derived from herpes simplex viruses (HSVs), including amplicon vectors, replication-defective HSV and attenuated HSV (Krisky et al., Gene Ther. 5:1517, 1998).

When a viral vector genome comprises a plurality of polynucleotides to be expressed in a host cell as separate transcripts, the viral vector may also comprise additional sequences between the two (or more) transcripts allowing for bicistronic or multicistronic expression. Examples of such sequences used in viral vectors include internal ribosome entry sites (IRES), furin cleavage sites, viral 2A peptide, or any combination thereof.

Other vectors developed for gene therapy can also be used with the compositions and methods of this disclosure. Such vectors include those derived from baculoviruses and α-viruses. (Jolly, D J. 1999. Emerging Viral Vectors. pp 209-40 in Friedmann T. ed. The Development of Human Gene Therapy. New York: Cold Spring Harbor Lab), or plasmid vectors (such as sleeping beauty or other transposon vectors).

Construction of an expression vector for producing a binding protein of this disclosure can be generated to obtain efficient transcription and translation. For example, a polynucleotide contained in a recombinant expression construct includes at least one appropriate expression control sequence (also called a regulatory sequence), such as a leader sequence and particularly a promoter operably (i.e., operatively) linked to the nucleotide sequence encoding the immunogen.

In certain embodiments, polynucleotides of the present disclosure are used to transfect/transduce a host cell (e.g., a T cell) for use in adoptive transfer therapy (e.g., targeting a cancer antigen). Methods for transfecting/transducing T cells with desired nucleic acids have been described (e.g., U.S. Patent Application Pub. No. US 2004/0087025) as have adoptive transfer procedures using T cells of desired target-specificity (e.g., Schmitt et al., Hum. Gen. 20:1240, 2009; Dossett et al., Mol. Ther. 17:742, 2009; Till et al., Blood 112:2261, 2008; Wang et al., Hum. Gene Ther. 18:712, 2007; Kuball et al., Blood 109:2331, 2007; US 2011/0243972; US 2011/0189141; Leen et al., Ann. Rev. Immunol. 25:243, 2007), such that adaptation of these methodologies to the presently disclosed embodiments is contemplated, based on the teachings herein, including those directed to binding proteins of the present disclosure. Accordingly, in another aspect, host cells are provided that comprise a polynucleotide of the present disclosure and express the encoded binding protein, wherein the encoded binding protein locates to the cell surface of the host cell when expressed. In certain embodiments, the host cell is a hematopoietic progenitor cell or a human immune system cell. In further embodiments, the immune system cell is a CD4+ T cell, a CD8+ T cell, a CD4− CD8− double negative T cell, a γδ T cell, a natural killer cell (e.g., NK cell or NK-T cell), a dendritic cell, or any combination thereof. In certain embodiments, the immune system cell is a CD4+ T cell. In certain embodiments, the T cell is a naïve T cell, a central memory T cell, a stem cell memory T cell, an effector memory T cell, or any combination thereof.

A host cell may include any individual cell or cell culture which may receive a vector or the incorporation of nucleic acids or express proteins. The term also encompasses progeny of the host cell, whether genetically or phenotypically the same or different. It will be appreciated that a polynucleotide encoding a binding protein of this disclosure is “heterologous” with regard to progeny of a host cell of the present disclosure, as well as to the host cell. Suitable host cells may depend on the vector and may include mammalian cells, animal cells, human cells, simian cells, insect cells, yeast cells, and bacterial cells. These cells may be induced to incorporate the vector or other material by use of a viral vector, transformation via calcium phosphate precipitation, DEAE-dextran, electroporation, microinjection, or other methods. See, for example, Sambrook et al., Molecular Cloning: A Laboratory Manual 2d ed. (Cold Spring Harbor Laboratory, 1989).

In any of the foregoing embodiments, an immune host cell may be modified to reduce or eliminate expression of one or more endogenous genes that encode a polypeptide involved in immune signaling or other related activities. Exemplary gene knockouts include those that encode PD-1, LAG-3, CTLA4, TIM3, an HLA molecule, a TCR molecule, or the like. Without wishing to be bound by theory, certain endogenously expressed immune cell proteins may be recognized as foreign by an allogeneic host receiving the modified immune cells, which may result in elimination of the modified immune cells (e.g., an HLA allele), or may downregulate the immune activity of the modified immune cells (e.g., PD-1, LAG-3, CTLA4), or may interfere with the binding activity of a heterologously expressed binding protein of the present disclosure (e.g., an endogenous TCR of a modified T cell that binds a non-KRAS antigen and thereby interferes with the modified immune cell binding a cell that expresses KRAS antigen).

Accordingly, decreasing or eliminating expression or activity of such endogenous genes or proteins can improve the activity, tolerance, or persistence of the modified immune cells in an autologous or allogeneic host setting, and may allow for universal administration of the cells (e.g., to any recipient regardless of HLA type). In certain embodiments, a modified immune cell is a donor cell (e.g., allogeneic) or an autologous cell. In certain embodiments, a modified immune cell of this disclosure comprises a chromosomal gene knockout of one or more of a gene that encodes PD-1, LAG-3, CTLA4, TIM3, TIGIT, an HLA component (e.g., a gene that encodes an α1 macroglobulin, an α2 macroglobulin, an α3 macroglobulin, a β1 microglobulin, or a β2 microglobulin), or a TCR component (e.g., a gene that encodes a TCR variable region or a TCR constant region) (see, e.g., Torikai et al., Nature Sci. Rep. 6:21757 (2016); Torikai et al., Blood 119(24):5697 (2012); and Torikai et al., Blood 122(8):1341 (2013), the gene-editing techniques, compositions, and adoptive cell therapies of which are herein incorporated by reference in their entirety).

As used herein, the term “chromosomal gene knockout” refers to a genetic alteration or introduced inhibitory agent in a host cell that prevents (e.g., reduces, delays, suppresses, or abrogates) production, by the host cell, of a functionally active endogenous polypeptide product. Alterations resulting in a chromosomal gene knockout can include, for example, introduced nonsense mutations (including the formation of premature stop codons), missense mutations, gene deletion, and strand breaks, as well as the heterologous expression of inhibitory nucleic acid molecules that inhibit endogenous gene expression in the host cell.

In certain embodiments, a chromosomal gene knock-out or gene knock-in is made by chromosomal editing of a host cell. Chromosomal editing can be performed using, for example, endonucleases. As used herein “endonuclease” refers to an enzyme capable of catalyzing cleavage of a phosphodiester bond within a polynucleotide chain. In certain embodiments, an endonuclease is capable of cleaving a targeted gene thereby inactivating or “knocking out” the targeted gene. An endonuclease may be a naturally occurring, recombinant, genetically modified, or fusion endonuclease. The nucleic acid strand breaks caused by the endonuclease are commonly repaired through the distinct mechanisms of homologous recombination or non-homologous end joining (NHEJ). During homologous recombination, a donor nucleic acid molecule may be used for a donor gene “knock-in”, for target gene “knock-out”, and optionally to inactivate a target gene through a donor gene knock in or target gene knock out event. NHEJ is an error-prone repair process that often results in changes to the DNA sequence at the site of the cleavage, e.g., a substitution, deletion, or addition of at least one nucleotide. NHEJ may be used to “knock-out” a target gene. Examples of endonucleases include zinc finger nucleases, TALE-nucleases, CRISPR-Cas nucleases, meganucleases, and megaTALs.

As used herein, a “zinc finger nuclease” (ZFN) refers to a fusion protein comprising a zinc finger DNA-binding domain fused to a non-specific DNA cleavage domain, such as a Fokl endonuclease. Each zinc finger motif of about 30 amino acids binds to about 3 base pairs of DNA, and amino acids at certain residues can be changed to alter triplet sequence specificity (see, e.g., Desjarlais et al., Proc. Natl. Acad. Sci. 90:2256-2260, 1993; Wolfe et al., J. Mol. Biol. 285:1917-1934, 1999). Multiple zinc finger motifs can be linked in tandem to create binding specificity to desired DNA sequences, such as regions having a length ranging from about 9 to about 18 base pairs. By way of background, ZFNs mediate genome editing by catalyzing the formation of a site-specific DNA double strand break (DSB) in the genome, and targeted integration of a transgene comprising flanking sequences homologous to the genome at the site of DSB is facilitated by homology directed repair. Alternatively, a DSB generated by a ZFN can result in knock out of target gene via repair by non-homologous end joining (NHEJ), which is an error-prone cellular repair pathway that results in the insertion or deletion of nucleotides at the cleavage site. In certain embodiments, a gene knockout comprises an insertion, a deletion, a mutation or a combination thereof, made using a ZFN molecule.

As used herein, a “transcription activator-like effector nuclease” (TALEN) refers to a fusion protein comprising a TALE DNA-binding domain and a DNA cleavage domain, such as a FokI endonuclease. A “TALE DNA binding domain” or “TALE” is composed of one or more TALE repeat domains/units, each generally having a highly conserved 33-35 amino acid sequence with divergent 12th and 13th amino acids. The TALE repeat domains are involved in binding of the TALE to a target DNA sequence. The divergent amino acid residues, referred to as the Repeat Variable Diresidue (RVD), correlate with specific nucleotide recognition. The natural (canonical) code for DNA recognition of these TALEs has been determined such that an HD (histine-aspartic acid) sequence at positions 12 and 13 of the TALE leads to the TALE binding to cytosine (C), NG (asparagine-glycine) binds to a T nucleotide, NI (asparagine-isoleucine) to A, NN (asparagine-asparagine) binds to a G or A nucleotide, and NG (asparagine-glycine) binds to a T nucleotide. Non-canonical (atypical) RVDs are also known (see, e.g., U.S. Patent Publication No. US 2011/0301073, which atypical RVDs are incorporated by reference herein in their entirety). TALENs can be used to direct site-specific double-strand breaks (DSB) in the genome of T cells. Non-homologous end joining (NHEJ) ligates DNA from both sides of a double-strand break in which there is little or no sequence overlap for annealing, thereby introducing errors that knock out gene expression. Alternatively, homology directed repair can introduce a transgene at the site of DSB providing homologous flanking sequences are present in the transgene. In certain embodiments, a gene knockout comprises an insertion, a deletion, a mutation or a combination thereof, and made using a TALEN molecule.

As used herein, a “clustered regularly interspaced short palindromic repeats/Cas” (CRISPR/Cas) nuclease system refers to a system that employs a CRISPR RNA (crRNA)-guided Cas nuclease to recognize target sites within a genome (known as protospacers) via base-pairing complementarity and then to cleave the DNA if a short, conserved protospacer associated motif (PAM) immediately follows 3′ of the complementary target sequence. CRISPR/Cas systems are classified into three types (i.e., type I, type II, and type III) based on the sequence and structure of the Cas nucleases. The crRNA-guided surveillance complexes in types I and III need multiple Cas subunits. Type II system, the most studied, comprises at least three components: an RNA-guided Cas9 nuclease, a crRNA, and a trans-acting crRNA (tracrRNA). The tracrRNA comprises a duplex forming region. A crRNA and a tracrRNA form a duplex that is capable of interacting with a Cas9 nuclease and guiding the Cas9/crRNA:tracrRNA complex to a specific site on the target DNA via Watson-Crick base-pairing between the spacer on the crRNA and the protospacer on the target DNA upstream from a PAM. Cas9 nuclease cleaves a double-stranded break within a region defined by the crRNA spacer. Repair by NHEJ results in insertions and/or deletions which disrupt expression of the targeted locus. Alternatively, a transgene with homologous flanking sequences can be introduced at the site of DSB via homology directed repair. The crRNA and tracrRNA can be engineered into a single guide RNA (sgRNA or gRNA) (see, e.g., Jinek et al., Science 337: 816-21, 2012). Further, the region of the guide RNA complementary to the target site can be altered or programed to target a desired sequence (Xie et al., PLOS One 9:e100448, 2014; U.S. Pat. Appl. Pub. No. US 2014/0068797, U.S. Pat. Appl. Pub. No. US 2014/0186843; U.S. Pat. No. 8,697,359, and PCT Publication No. WO 2015/071474; each of which is incorporated by reference). In certain embodiments, a gene knockout comprises an insertion, a deletion, a mutation or a combination thereof, and made using a CRISPR/Cas nuclease system. Exemplary gRNA sequences and methods of using the same to knock out endogenous genes that encode immune cell proteins include those described in Ren et al., Clin. Cancer Res. 23(9):2255-2266 (2017), the gRNAs, CAS9 DNAs, vectors, and gene knockout techniques of which are hereby incorporated by reference in their entirety.

As used herein, a “meganuclease,” also referred to as a “homing endonuclease,” refers to an endodeoxyribonuclease characterized by a large recognition site (double stranded DNA sequences of about 12 to about 40 base pairs). Meganucleases can be divided into five families based on sequence and structure motifs: LAGLIDADG (SEQ ID NO:159), GIY-YIG (SEQ ID NO:160), HNH, His-Cys box and PD-(D/E)XK (SEQ ID NO:161). Exemplary meganucleases include I-SceI, I-CeuI, PI-Pspl, PI-Sce, I-SceIV, I-CsmI, I-PanI, I-SceII, I-PpoI, I-SceIII, I-CreI, I-TevI, I-TevII and I-TevIII, whose recognition sequences are known (see, e.g., U.S. Pat. Nos. 5,420,032 and 6,833,252; Belfort et al., Nucleic Acids Res. 25:3379-3388, 1997; Dujon et al., Gene 82:115-118, 1989; Perler et al., Nucleic Acids Res. 22:1125-1127, 1994; Jasin, Trends Genet. 12:224-228, 1996; Gimble et al., J. Mol. Biol. 263:163-180, 1996; Argast et al., J. Mol. Biol. 280:345-353, 1998).

In certain embodiments, naturally-occurring meganucleases may be used to promote site-specific genome modification of a target selected from PD-1, LAG3, TIM3, CTLA4, TIGIT, an HLA-encoding gene, or a TCR component-encoding gene. In other embodiments, an engineered meganuclease having a novel binding specificity for a target gene is used for site-specific genome modification (see, e.g., Porteus et al., Nat. Biotechnol. 23:967-73, 2005; Sussman et al., J. Mol. Biol. 342:31-41, 2004; Epinat et al., Nucleic Acids Res. 31:2952-62, 2003; Chevalier et al., Molec. Cell 10:895-905, 2002; Ashworth et al., Nature 441:656-659, 2006; Paques et al., Curr. Gene Ther. 7:49-66, 2007; U.S. Patent Publication Nos. US 2007/0117128; US 2006/0206949; US 2006/0153826; US 2006/0078552; and US 2004/0002092). In further embodiments, a chromosomal gene knockout is generated using a homing endonuclease that has been modified with modular DNA binding domains of TALENs to make a fusion protein known as a megaTAL. MegaTALs can be utilized to not only knock-out one or more target genes, but to also introduce (knock in) heterologous or exogenous polynucleotides when used in combination with an exogenous donor template encoding a polypeptide of interest.

In certain embodiments, a chromosomal gene knockout comprises an inhibitory nucleic acid molecule that is introduced into a host cell (e.g., an immune cell) comprising a heterologous polynucleotide encoding an antigen-specific receptor that specifically binds to a tumor associated antigen, wherein the inhibitory nucleic acid molecule encodes a target-specific inhibitor and wherein the encoded target-specific inhibitor inhibits endogenous gene expression (i.e., of PD-1, TIM3, LAG3, CTLA4, TIGIT, an HLA component, or a TCR component, or any combination thereof) in the host immune cell.

A chromosomal gene knockout can be confirmed directly by DNA sequencing of the host immune cell following use of the knockout procedure or agent. Chromosomal gene knockouts can also be inferred from the absence of gene expression (e.g., the absence of an mRNA or polypeptide product encoded by the gene) following the knockout.

In another aspect, compositions are provided herein that comprise a modified immune cell of the present disclosure and a pharmaceutically acceptable carrier, diluent, or excipient. Also provided herein are unit doses that comprise an effective amount of a modified immune cell or of a composition comprising the modified immune cell. In certain embodiments, a unit dose comprises (i) a composition comprising at least about 30%, at least about 40%, at least about 50%, at least about 60%, at least about 70%, at least about 80%, at least about 85%, at least about 90%, or at least about 95% modified CD4⁺ T cells, combined with (ii) a composition comprising at least about 30%, at least about 40%, at least about 50%, at least about 60%, at least about 70%, at least about 80%, at least about 85%, at least about 90%, or at least about 95% modified CD8⁺ T cells, in about a 1:1 ratio, which, in some embodiments, contains a reduced amount or substantially no naïve T cells (i.e., has less than about 50%, less than about 40%, less than about 30%, less then about 20%, less than about 10%, less than about 5%, or less then about 1% the population of naïve T cells present in a unit dose as compared to a patient sample having a comparable number of PBMCs).

In some embodiments, a unit dose comprises (i) a composition comprising at least about 50% modified CD4⁺ T cells, combined with (ii) a composition comprising at least about 50% modified CD8⁺ T cells, in about a 1:1 ratio, wherein the unit dose contains a reduced amount or substantially no naïve T cells. In further embodiments, a unit dose comprises (i) a composition comprising at least about 60% modified CD4⁺ T cells, combined with (ii) a composition comprising at least about 60% modified CD8⁺ T cells, in about a 1:1 ratio, wherein the unit dose contains a reduced amount or substantially no naïve T cells. In still further embodiments, a unit dose comprises (i) a composition comprising at least about 70% engineered CD4⁺ T cells, combined with (ii) a composition comprising at least about 70% engineered CD8⁺ T cells, in about a 1:1 ratio, wherein the unit dose contains a reduced amount or substantially no naïve T cells. In some embodiments, a unit dose comprises (i) a composition comprising at least about 80% modified CD4⁺ T cells, combined with (ii) a composition comprising at least about 80% modified CD8⁺ T cells, in about a 1:1 ratio, wherein the unit dose contains a reduced amount or substantially no naïve T cells. In some embodiments, a unit dose comprises (i) a composition comprising at least about 85% modified CD4⁺ T cells, combined with (ii) a composition comprising at least about 85% modified CD8⁺ T cells, in about a 1:1 ratio, wherein the unit dose contains a reduced amount or substantially no naïve T cells. In some embodiments, a unit dose comprises (i) a composition comprising at least about 90% modified CD4⁺ T cells, combined with (ii) a composition comprising at least about 90% modified CD8⁺ T cells, in about a 1:1 ratio, wherein the unit dose contains a reduced amount or substantially no naïve T cells.

In any of the embodiments described herein, a unit dose can comprise equal, or approximately equal numbers of modified CD45RA⁻ CD3⁺ CD8⁺ and modified CD45RA⁻ CD3⁺ CD4⁺ T_{M} cells.

In some embodiments, a dose comprises up to or at least about 3.3×10⁵ modified T cells/kg (patient body weight), up to or at least about 1×10⁶ modified T cells/kg, up to or least about 3.3×10⁶ modified T cells/kg, up to or at least about 1×10⁷ modified T cells/kg, or more. Doses of modified immune cells for treating diseases such as cancer are described further herein.

### Lymphodepletion Chemotherapy

In some embodiments, a modified immune cell of the present disclosure (e.g., a modified T cell) is administered to a subject who previously received lymphodepletion chemotherapy. As used herein, the term “chemotherapeutic agent” (which may also be called a “chemotherapeutic” or a “chemotherapy” herein) refers to a chemical agent, drug, or other therapeutic modality that targets diseased cells (e.g., cancer cells) for inhibition or death. Chemotherapeutic agents of the present disclosure encompass different structures, forms, and systems of delivery, and are to be understood in terms of their functionality for inhibiting or killing diseased cells.

Lymphocytes may be depleted using irradiation or chemotherapy to kill lymphocytes, reduce tumor burden, or facilitate survival of subsequently transferred modified immune cells of the present disclosure. In some embodiments, lymphodepletion chemotherapy comprises an alkylating agent, e.g., cyclophosphamide. In further embodiments, the subject has received cyclophosphamide administered at about 100, 125, 150, 175, 200, 225, 250, 275, 300, 350, 0.375, 400, 425, 450, 475, or about 500 mg/m². In some embodiments, the subject has received cyclophosphamide at about 300 mg/m². In any of the herein disclosed embodiments, lymphodepletion can comprise a platin (e.g., oxaliplatin), fludarabine (optionally administered at about 10, 15, 20, 25, 30, 35, 40, 45, or 50 mg/m²), or both, or in combination with an alkylating agent such as cyclophosphamide.

In some embodiments, the subject has received cyclophosphamide and fludarabine. In some embodiments, the subject has received oxaliplatin and cyclophosphamide. In some embodiments, the subject has received cyclophosphamide, fludarabine, and oxaliplatin, which may be in any combination or combinations; e.g., in one combination comprising fludarabine and cyclophosphamide and another combination comprising oxaliplatin and cyclophosphamide.

In particular embodiments, the subject has received lymphodepletion chemotherapy comprising cyclophosphamide at about 300 mg/m² and fludarabine at about 30 mg/m².

Other chemotherapeutic agents are described herein and may be used in any combination with a modified immune cell of this disclosure, with or without a lymphodepletion chemotherapy, or as a secondary therapy.

In certain aspects, a modified immune cell of the present disclosure is used with an with an inhibitor of an immune suppression component or an agonist of a stimulatory immune checkpoint molecule, as described herein, to enhance an antitumor response by the immune system and to, ultimately, treat a tumor or associated cancer.

As used herein, the term “immune suppression component” or “immunosuppression component” refers to one or more cells, proteins, molecules, compounds or complexes providing inhibitory signals to assist in controlling or suppressing an immune response. For example, immune suppression components include those molecules that partially or totally block immune stimulation; decrease, prevent or delay immune activation; or increase, activate, or up regulate immune suppression. Exemplary immunosuppression component targets are described in further detail herein and include PD-1, PD-L1, CTLA4, Tim-3, LAG-3, TIGIT, or any combination thereof.

An inhibitor of an immune suppression component may be a compound, an antibody, an antibody fragment or fusion polypeptide (e.g., Fc fusion, such as CTLA4-Fc or LAG3-Fc), an antisense molecule, a ribozyme or RNAi molecule, or a low molecular weight organic molecule. In any of the embodiments disclosed herein, a method may comprise administering a modified immune cell with one or more inhibitor of any one of the following immune suppression components, singly or in any combination.

### Agonists of Stimulatory Immune Checkpoint Molecules

In certain embodiments, a modified human immune cell is used in combination (e.g., concurrently, sequentially, or simultaneously) with an agent that increases the activity (i.e., is an agonist) of a stimulatory immune checkpoint molecule. For example, a modified immune cell can be used in combination with a CD137 (4-1BB) agonist (such as, for example, urelumab), a CD134 (OX-40) agonist (such as, for example, MEDI6469, MEDI6383, or MEDI0562), lenalidomide, pomalidomide, a CD27 agonist (such as, for example, CDX-1127), a CD28 agonist (such as, for example, TGN1412, CD80, or CD86), a CD40 agonist (such as, for example, CP-870,893, rhuCD40L, or SGN-40), a CD122 agonist (such as, for example, IL-2), an agonist of GITR (such as, for example, humanized monoclonal antibodies described in PCT Patent Publication No. WO 2016/054638), or an agonist of ICOS (CD278) (such as, for example, GSK3359609, mAb 88.2, JTX-2011, Icos 145-1, or Icos 314-8), or any combination thereof. In any of the embodiments disclosed herein, a method may comprise administering a modified human immune cell with one or more agonist of a stimulatory immune checkpoint molecule, including any of the foregoing, singly or in any combination.

### Secondary Therapies

In other embodiments, methods of the present disclosure further comprise administering a secondary therapy comprising one or more of: an antibody or antigen binding fragment specific for a cancer antigen expressed by the solid tumor being targeted; a chemotherapeutic agent; surgery; radiation therapy treatment; a cytokine; an RNA interference therapy, or any combination thereof.

Exemplary monoclonal antibodies useful in cancer therapies are known in the art and include, for example, monoclonal antibodies described in Galluzzi et al., Oncotarget 5(24):12472-12508, 2014, which monoclonal antibodies and cancer therapies using the same are incorporated herein by reference.

In certain embodiments, a combination therapy method comprises administering a modified human immune cell and further administering a radiation treatment or a surgery. Radiation therapy includes X-ray therapies, such as gamma-irradiation, and radiopharmaceutical therapies. Surgeries and surgical techniques appropriate to treating a given cancer or non-inflamed solid tumor in a subject are known to those of ordinary skill in the art.

In certain embodiments, a combination therapy comprises administering a modified human immune cell and further administering a chemotherapeutic agent. A chemotherapeutic agent includes, but is not limited to, an inhibitor of chromatin function, a topoisomerase inhibitor, a microtubule inhibiting drug, a DNA damaging agent, an antimetabolite (such as folate antagonists, pyrimidine analogs, purine analogs, and sugar-modified analogs), a DNA synthesis inhibitor, a DNA interactive agent (such as an intercalating agent), and a DNA repair inhibitor. Illustrative chemotherapeutic agents include, without limitation, the following groups: anti-metabolites/anti-cancer agents, such as pyrimidine analogs (5-fluorouracil, floxuridine, capecitabine, gemcitabine and cytarabine) and purine analogs, folate antagonists and related inhibitors (mercaptopurine, thioguanine, pentostatin and 2-chlorodeoxyadenosine (cladribine)); antiproliferative/antimitotic agents including vinca alkaloids (vinblastine, vincristine, and vinorelbine), microtubule disruptors such as taxane (paclitaxel, docetaxel), vincristin, vinblastin, nocodazole, epothilones and navelbine, epidipodophyllotoxins (etoposide, teniposide), DNA damaging agents (actinomycin, amsacrine, anthracyclines, bleomycin, busulfan, camptothecin, carboplatin, chlorambucil, cisplatin, cyclophosphamide, cytoxan, dactinomycin, daunorubicin, doxorubicin, epirubicin, hexamethylmelamineoxaliplatin, iphosphamide, melphalan, merchlorehtamine, mitomycin, mitoxantrone, nitrosourea, plicamycin, procarbazine, taxol, taxotere, temozolamide, teniposide, triethylenethiophosphoramide and etoposide (VP 16)); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine (BCNU) and analogs, streptozocin), trazenes-dacarbazinine (DTIC); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory agents; antisecretory agents (breveldin); immunosuppressives (cyclosporine, tacrolimus (FK-506), sirolimus (rapamycin), azathioprine, mycophenolate mofetil); anti-angiogenic compounds (TNP470, genistein) and growth factor inhibitors (vascular endothelial growth factor (VEGF) inhibitors, fibroblast growth factor (FGF) inhibitors); angiotensin receptor blocker; nitric oxide donors; anti-sense oligonucleotides; antibodies (trastuzumab, rituximab); chimeric antigen receptors; cell cycle inhibitors and differentiation inducers (tretinoin); mTOR inhibitors, topoisomerase inhibitors (doxorubicin (adriamycin), amsacrine, camptothecin, daunorubicin, dactinomycin, eniposide, epirubicin, etoposide, idarubicin, irinotecan (CPT-11) and mitoxantrone, topotecan, irinotecan), corticosteroids (cortisone, dexamethasone, hydrocortisone, methylpednisolone, prednisone, and prenisolone); growth factor signal transduction kinase inhibitors; mitochondrial dysfunction inducers, toxins such as Cholera toxin, ricin, Pseudomonas exotoxin, Bordetella pertussis adenylate cyclase toxin, or diphtheria toxin, and caspase activators; and chromatin disruptors. In some embodiments, chemotherapy comprises a lymphodepleting chemotherapy agent as described herein.

Cytokines can be used to manipulate host immune response towards anticancer activity. See, e.g., Floros & Tarhini, Semin. Oncol. 42(4):539-548, 2015. Cytokines useful for promoting immune anticancer or antitumor response include, for example, IFN-α, IL-2, IL-3, IL-4, IL-10, IL-12, IL-13, IL-15, IL-16, IL-17, IL-18, IL-21, IL-24, and GM-CSF, singly or in any combination.

Another cancer therapy approach involves reducing expression of oncogenes and other genes needed for growth, maintenance, proliferation, and immune evasion by cancer cells. RNA interference, and in particular the use of microRNAs (miRNAs) small inhibitory RNAs (siRNAs) provides an approach for knocking down expression of cancer genes. See, e.g., Larsson et al., Cancer Treat. Rev. 16(55):128-135, 2017. Techniques for making and using RNA for cancer therapy are known to those having ordinary skill in the art.

In any of the embodiments disclosed herein, any of the therapeutic agents (e.g., a modified immune cell, an inhibitor of an immune suppression component, an agonist of a stimulatory immune checkpoint molecule, an antitumor lymphocyte, a chemotherapeutic agent, a radiation therapy, a surgery, a cytokine, or an inhibitory RNA) may be administered once or more than once to the subject over the course of a treatment, and, in combinations, may be administered to the subject in any order (e.g., simultaneously, concurrently, or in any sequence) or any combination. An appropriate dose, suitable duration, and frequency of administration of the compositions will be determined by such factors as a condition of the patient; size, type, spread, growth, and severity of the tumor or cancer; particular form of the active ingredient; and the method of administration.

In certain embodiments, a plurality of doses of a modified immune cell as described herein is administered to the subject, which may be administered at intervals between administrations of about two to about four weeks. In further embodiments, a cytokine (e.g., IL-2, IL-15, IL-21) is administered sequentially, provided that the subject was administered the recombinant host cell at least three or four times before cytokine administration. In certain embodiments, the cytokine is administered concurrently with the host cell. In certain embodiments, the cytokine is administered subcutaneously.

In still further embodiments, the subject being treated is further receiving immunosuppressive therapy, such as calcineurin inhibitors, corticosteroids, microtubule inhibitors, low dose of a mycophenolic acid prodrug, or any combination thereof. In yet further embodiments, the subject being treated has received a non-myeloablative or a myeloablative hematopoietic cell transplant, wherein the treatment may be administered at least two to at least three months after the non-myeloablative hematopoietic cell transplant.

### Uses

Compositions of the present disclosure are useful in treating diseases or conditions. In certain embodiments, a disease comprises a proliferative disease, such as a hyperproliferative disease. Exemplary proliferative disorders include tumors, cancers, neoplastic tissue, carcinoma, sarcoma, malignant cells, pre-malignant cells, as well as non-neoplastic or non-malignant hyperproliferative disorders (e.g., adenoma, fibroma, lipoma, leiomyoma, hemangioma, fibrosis, restenosis, as well as autoimmune diseases such as rheumatoid arthritis, osteoarthritis, psoriasis, inflammatory bowel disease, or the like).

Furthermore, “cancer” may refer to any accelerated or dysregulated proliferation of cells, including solid tumors, ascites tumors, blood or lymph or other malignancies; connective tissue malignancies; metastatic disease; minimal residual disease following transplantation of organs or stem cells; multi-drug resistant cancers, primary or secondary malignancies, angiogenesis related to malignancy, or other forms of cancer. Exemplary cancers treatable according to presently disclosed methods and compositions include both those characterized by solid tumors (e.g., triple negative breast cancer (TNBC), non small-cell lung cancer (NSCLC)) and hematological malignancies (e.g., ALL, CLL, and MCL).

In general, cancers treatable by presently disclosed methods and compositions include carcinomas, sarcomas, gliomas, lymphomas, leukemias, myelomas, cancers of the head or neck, melanoma, pancreatic cancer, cholangiocarcinoma, hepatocellular cancer, breast cancer, gastric cancer, non-small-cell lung cancer, prostate cancer, esophageal cancer, mesothelioma, small-cell lung cancer, colorectal cancer, glioblastoma, Askin's tumor, sarcoma botryoides, chondrosarcoma, Ewing's sarcoma, PNET, malignant hemangioendothelioma, malignant schwannoma, osteosarcoma, alveolar soft part sarcoma, angiosarcoma, cystosarcoma phyllodes, dermatofibrosarcoma protuberans (DFSP), desmoid tumor, desmoplastic small round cell tumor, epithelioid sarcoma, extraskeletal chondrosarcoma, extraskeletal osteosarcoma, fibrosarcoma, gastrointestinal stromal tumor (GIST), hemangiopericytoma, hemangiosarcoma, Kaposi's sarcoma, leiomyosarcoma, liposarcoma, lymphangiosarcoma, lymphosarcoma, undifferentiated pleomorphic sarcoma, malignant peripheral nerve sheath tumor (MPNST), neurofibrosarcoma, rhabdomyosarcoma, synovial sarcoma, undifferentiated pleomorphic sarcoma, squamous cell carcinoma, basal cell carcinoma, adenocarcinoma, linitis plastic, vipoma, cholangiocarcinoma, hepatocellular carcinoma, adenoid cystic carcinoma, renal cell carcinoma, Grawitz tumor, ependymoma, astrocytoma, oligodendroglioma, brainstem glioma, optice nerve glioma, a mixed glioma, Hodgkin's lymphoma, a B-cell lymphoma, non-Hodgkin's lymphoma (NHL), Burkitt's lymphoma, small lymphocytic lymphoma (SLL), diffuse large B-cell lymphoma, follicular lymphoma, immunoblastic large cell lymphoma, precursor B-lymphoblastic lymphoma, and mantle cell lymphoma, Waldenström's macroglobulinemia, CD37+ dendritic cell lymphoma, lymphoplasmacytic lymphoma, splenic marginal zone lymphoma, extra-nodal marginal zone B-cell lymphoma of mucosa-associated (MALT) lymphoid tissue, nodal marginal zone B-cell lymphoma, mediastinal (thymic) large B-cell lymphoma, intravascular large B-cell lymphoma, primary effusion lymphoma, adult T-cell lymphoma, extranodal NK/T-cell lymphoma, nasal type, enteropathy-associated T-cell lymphoma, hepatosplenic T-cell lymphoma, blastic NK cell lymphoma, Sezary syndrome, angioimmunoblastic T cell lymphoma, anaplastic large cell lymphoma, chondrosarcoma; fibrosarcoma (fibroblastic sarcoma); Dermatofibrosarcoma protuberans (DFSP); osteosarcoma; rhabdomyosarcoma; Ewing's sarcoma; a gastrointestinal stromal tumor; Leiomyosarcoma; angiosarcoma (vascular sarcoma); Kaposi's sarcoma; liposarcoma; pleomorphic sarcoma; or synovial sarcoma; lung carcinoma (e.g., Adenocarcinoma, Squamous Cell Carcinoma (Epidermoid Carcinoma); Squamous cell carcinoma; Adenocarcinoma; Adenosquamous carcinoma; anaplastic carcinoma; Large cell carcinoma; Small cell carcinoma; a breast carcinoma (e.g., Ductal Carcinoma in situ (non-invasive), Lobular carcinoma in situ (non-invasive), Invasive Ductal Carcinoma, Invasive lobular carcinoma, Non-invasive Carcinoma); a liver carcinoma (e.g., Hepatocellular Carcinoma, Cholangiocarcinomas or Bile Duct Cancer); Large-cell undifferentiated carcinoma, Bronchioalveolar carcinoma); an ovarian carcinoma (e.g., Surface epithelial-stromal tumor (Adenocarcinoma) or ovarian epithelial carcinoma (which includes serous tumor, endometrioid tumor and mucinous cystadenocarcinoma), Epidermoid (Squamous cell carcinoma), Embryonal carcinoma and choriocarcinoma (germ cell tumors)); a kidney carcinoma (e.g., Renal adenocarcinoma, hypernephroma, Transitional cell carcinoma (renal pelvis), Squamous cell carcinoma, Bellini duct carcinoma, Clear cell adenocarcinoma, Transitional cell carcinoma, Carcinoid tumor of the renal pelvis); an adrenal carcinoma (e.g., Adrenocortical carcinoma), a carcinoma of the testis (e.g., Germ cell carcinoma (Seminoma, Choriocarcinoma, Embryonal carciroma, Teratocarcinoma), Serous carcinoma); Gastric carcinoma (e.g., Adenocarcinoma); an intestinal carcinoma (e.g., Adenocarcinoma of the duodenum); a colorectal carcinoma; or a skin carcinoma (e.g., Basal cell carcinoma, Squamous cell carcinoma); ovarian carcinoma, an ovarian epithelial carcinoma, a cervical adenocarcinoma or small cell carcinoma, a pancreatic carcinoma, a colorectal carcinoma (e.g., an adenocarcinoma or squamous cell carcinoma), a lung carcinoma, a breast ductal carcinoma, or an adenocarcinoma of the prostate.

Subjects that can be treated by the present invention are, in general, human and other primate subjects, such as monkeys and apes for veterinary medicine purposes. In any of the aforementioned embodiments, the subject may be a human subject. The subjects can be male or female and can be any suitable age, including infant, juvenile, adolescent, adult, and geriatric subjects. Cells according to the present disclosure may be administered in a manner appropriate to the disease, condition, or disorder to be treated as determined by persons skilled in the medical art. In any of the above embodiments, a cell comprising a binding protein as described herein is administered intravenously, intraperitoneally, intratumorally, into the bone marrow, into a lymph node, or into the cerebrospinal fluid. An appropriate dose, suitable duration, and frequency of administration of the compositions will be determined by such factors as a condition of the patient; size, type, and severity of the disease, condition, or disorder; the undesired type or level or activity of the immunotherapy cells, the particular form of the active ingredient; and the method of administration.

In any of the above embodiments, methods of the present disclosure comprise administering a therapeutically effective amount of a host cell expressing a binding protein of the present disclosure.

An effective amount of a therapeutic or pharmaceutical composition refers to an amount sufficient, at dosages and for periods of time needed, to achieve the desired clinical results or beneficial treatment, as described herein. An effective amount may be delivered in one or more administrations. If the administration is to a subject already known or confirmed to have a disease or disease-state, the term “therapeutic amount” may be used in reference to treatment, whereas “prophylactically effective amount” may be used to describe administrating an effective amount to a subject that is susceptible or at risk of developing a disease or disease-state (e.g., recurrence) as a preventative course.

A “therapeutically effective amount” or “effective amount” of a binding protein, or host cell expressing a binding protein of this disclosure refers to an amount of binding proteins or host cells sufficient to result in a therapeutic effect, including improved clinical outcome; lessening or alleviation of symptoms associated with a disease; decreased occurrence of symptoms; improved quality of life; longer disease-free status; diminishment of extent of disease, stabilization of disease state; delay of disease progression; remission; survival; or prolonged survival in a statistically significant manner. When referring to an individual active ingredient or a cell expressing a single active ingredient, administered alone, a therapeutically effective amount refers to the effects of that ingredient or cell expressing that ingredient alone. When referring to a combination, a therapeutically effective amount refers to the combined amounts of active ingredients or combined adjunctive active ingredient with a cell expressing an active ingredient that results in a therapeutic effect, whether administered serially or simultaneously. A combination may also be a cell expressing more than one active ingredient, such as two different binding proteins (e.g., CARs) that each specifically bind to a target (e.g., each binding to the same or to a different hyperproliferative disease-associated antigen) or a binding protein-modified immune cell, a chemotherapeutic agent, or another relevant therapeutic.

A therapeutically effective amount of cells in a composition is at least one cell (for example, one binding protein modified CD8+ T cell subpopulation; one binding protein modified CD4+ T cell subpopulation) or is, in certain embodiments, greater than 10² cells, for example, up to 10⁶, up to 10⁷, up to 10⁸ cells, up to 10⁹ cells, or more than 10¹⁰ cells. In certain embodiments, the cells are administered in a range from about 10⁵ to about 10¹⁰ cells/m², and in some embodiments in a range of about 10⁶ to about 10⁹ cells/m². In certain embodiments, a composition comprises binding protein-modified CD4+ T cells and binding protein-modified CD8+ T cells in about a 1:1 ratio; e.g., 50% CD4+ T cells and 50% CD8+ T cells, +/−20%, or +/−15%, or +/−10%, or +/−5%, or +/−4%, or +/−3%, or +/−2%, or +/−1%.

The number of cells will depend upon the ultimate use for which the composition is intended as well the type of cells included therein. For example, cells modified to contain a binding protein specific for a particular antigen will comprise a cell population containing at least 30%, 35%, 40%, 45%, 50%, 55%, 60%, 65%, 70%, 75%, 80%, 85%, 90%, 95% or more of such cells. For uses provided herein, cells are generally in a volume of a liter or less, 500 mls or less, 250 mls or less, or 100 mls or less. In embodiments, the density of the desired cells is typically greater than 10⁴ cells/ml and generally is greater than 10⁷ cells/ml, generally 10⁸ cells/ml or greater. The cells may be administered as a single infusion or in multiple infusions over a range of time. A clinically relevant number of immune cells can be apportioned into multiple infusions that cumulatively equal or exceed 10⁴, 10⁵, 10⁶, 10⁷, 10⁸, 10⁹, 10¹⁰, or 10¹¹ cells.

In certain embodiments, doses provided herein comprise a therapeutically effective amount of the host cells (e.g., modified immune cells comprising a polynucleotide of the present disclosure). In some embodiments, a dose comprises up to or at least about 3.3×10⁵ modified T cells/kg (patient body weight), up to or at least about 1×10⁶ modified T cells/kg, up to or least about 3.3×10⁶ modified T cells/kg, up to or at least about 1×10⁷ modified T cells/kg, or more. In some embodiments, a dose comprises (a) about 1×10⁵ modified cells/kg; (b) about 3.3×10⁵ modified cells/kg; (c) about 1×10⁶ modified cells/kg; (d) about 3.3×10⁶ modified cells/kg; or (e) about 1×10⁷ modified cells/kg. In some embodiments, a dose comprises at least about 3.3×10⁵ modified cells/kg, at least about 1×10⁶ modified immune cells/kg, or at least about 3.3×10⁶ modified cells/kg.

Also contemplated are pharmaceutical compositions that comprise binding proteins or cells expressing the binding proteins as disclosed herein and a pharmaceutically acceptable carrier, diluents, or excipient. The term “pharmaceutically acceptable excipient or carrier” or “physiologically acceptable excipient or carrier” refer to biologically compatible vehicles, e.g., physiological saline, which are described in greater detail herein, that are suitable for administration to a human or other non-human mammalian subject and generally recognized as safe or not causing a serious adverse event. Suitable excipients include water, saline, dextrose, glycerol, or the like and combinations thereof. In embodiments, compositions comprising binding proteins or host cells as disclosed herein further comprise a suitable infusion media. Suitable infusion media can be any isotonic medium formulation, typically normal saline, Normosol R (Abbott) or Plasma-Lyte A (Baxter), 5% dextrose in water, Ringer's lactate can be utilized. An infusion medium can be supplemented with human serum albumin or other human serum components.

Pharmaceutical compositions may be administered in a manner appropriate to the disease or condition to be treated (or prevented) as determined by persons skilled in the medical art. An appropriate dose and a suitable duration and frequency of administration of the compositions will be determined by such factors as the health condition of the patient, size of the patient (i.e., weight, mass, or body area), the type and severity of the patient's condition, the undesired type or level or activity of the tagged immunotherapy cells, the particular form of the active ingredient, and the method of administration. In general, an appropriate dose and treatment regimen provide the composition(s) in an amount sufficient to provide therapeutic and/or prophylactic benefit (such as described herein, including an improved clinical outcome, such as more frequent complete or partial remissions, or longer disease-free and/or overall survival, or a lessening of symptom severity). For prophylactic use, a dose should be sufficient to prevent, delay the onset of, or diminish the severity of a disease associated with disease or disorder. Prophylactic benefit of the compositions administered according to the methods described herein can be determined by performing pre-clinical (including in vitro and in vivo animal studies) and clinical studies and analyzing data obtained therefrom by appropriate statistical, biological, and clinical methods and techniques, all of which can readily be practiced by a person skilled in the art.

Certain methods of treatment or prevention contemplated herein include administering a host cell (which may be autologous, allogeneic or syngeneic) comprising a desired polynucleotide as described herein that is stably integrated into the chromosome of the cell. For example, such a cellular composition may be generated ex vivo using autologous, allogeneic or syngeneic immune system cells (e.g., T cells, antigen-presenting cells, natural killer cells) in order to administer a desired, binding protein-expressing T-cell composition to a subject as an adoptive immunotherapy. In certain embodiments, the host cell is a hematopoietic progenitor cell or a human immune cell. In certain embodiments, the immune system cell is a CD4+ T cell, a CD8+ T cell, a CD4− CD8− double negative T cell, a γδ T cell, a natural killer cell, a dendritic cell, or any combination thereof. In certain embodiments, the immune system cell is a naïve T cell, a central memory T cell, an effector memory T cell, a stem cell memory T cell, or any combination thereof.

As used herein, administration of a composition or therapy refers to delivering the same to a subject, regardless of the route or mode of delivery. Administration may be effected continuously or intermittently, and parenterally. Administration may be for treating a subject already confirmed as having a recognized condition, disease or disease state, or for treating a subject susceptible to or at risk of developing such a condition, disease or disease state. Co-administration with an adjunctive therapy may include simultaneous and/or sequential delivery of multiple agents in any order and on any dosing schedule (e.g., binding protein-expressing recombinant (i.e., engineered) host cells with one or more cytokines; immunosuppressive therapy such as calcineurin inhibitors, corticosteroids, microtubule inhibitors, low dose of a mycophenolic acid prodrug, or any combination thereof).

Lymphodepletion chemotherapy agents are used as pre-conditioning agents in some adoptive cell therapies and, in some cases, are administered to a subject prior to the subject receiving a cell therapy. In some embodiments of the present disclosure, a subject receives and completes a lymphodepletion chemotherapy treatment (such as any of the lymphodepletion chemotherapies disclosed herein, including cyclophosphamide, fludarabine, oxaliplatin, or any combination or combinations thereof) at least about 12 hours, at least about 24 hours, at least about 36 hours, at least about 48 hours, at least about 60 hours, at least about 72 hours, at least about 84 hours, at least about 96 hours, or more, prior to receiving a dose of a modified immune cell of this disclosure. In further embodiments, a subject administered a dose of a modified immune cell of this disclosure had previously been administered lymphodepleting chemotherapy about 36 to about 96 hours prior to being administered the dose. It will be understood that in embodiments wherein a subject receives a first dose of a modified immune cell and a subsequent second dose of the modified immune cell, as described herein, a lymphodepletion chemotherapy may be administered before, concurrent with, simultaneous with, or after either or both of the first and second dose of the modified immune cell.

In certain embodiments, a plurality of doses of a modified immune cell (e.g., a T cell) as described herein are administered to the subject, which may, in some embodiments, be administered at intervals between administrations of about two to about four weeks. In some embodiments, a first dose is provided, and a second dose is provided from about 21 to about 28 days thereafter.

The compositions administered in first and second or subsequent doses may be the same or different in terms of, for example, the concentration of cells, the type of cells (e.g., CD4+ or CD8+), or both. In certain embodiments, a second dose comprises a composition comprising binding protein-modified CD4+ T cells and binding protein-modified CD8+ T cells in about a 1:1 ratio; e.g., 50% CD4+ T cells and 50% CD8+ T cells, +/−20%, or +/−15%, or +/−10%, or +/−5%, or +/−4%, or +/−3%, or +/−2%, or +/−1%. In some embodiments comprising administration of a modified immune cell of this disclosure, a second dose comprises (a) about 1×10⁵ modified cells/kg; (b) about 3.3×10⁵ modified cells/kg; (c) about 1×10⁶ modified cells/kg; (d) about 3.3×10⁶ modified cells/kg; or (e) about 1×10⁷ modified cells/kg. In some embodiments comprising a modified immune cell of this disclosure, a second dose comprises a same amount of a modified immune cell as compared to the first dose. In other embodiments comprising modified T cells of this disclosure, a second dose comprises a reduced amount of a modified immune cell as compared to the first dose. In still other embodiments comprising a modified immune cell of this disclosure, a second dose comprises an increased amount of the modified immune cell as compared to the first dose. In particular embodiments, a first dose comprises about 1×10⁶ of a modified T cell/kg and the second dose comprises about 3.3×10⁶ of the modified T cell/kg. In other embodiments, a first dose comprises about 3.3×10⁶ of a modified T cell/kg and the second dose comprises about 1.0×10⁷ of the modified T cell/kg. In other embodiments, a first dose comprises about 1×10⁶ of a modified T cell/kg and the second dose comprises about 1.0×10⁷ of the modified T cell/kg. It will be appreciated that each of a first and second dose of a modified immune cell of this disclosure can independently comprise any of the doses enumerated herein, or any dose therebetween.

In any of the embodiments disclosed herein, a dose (e.g., either or both of a first dose and a second dose, or any subsequent dose) may be administered to the subject over a period from about 1 minute to about 1 hour, or from about 5 minutes to about 50 minutes, or from about 10 minutes to about 40 minutes, or from about 20 minutes to about 30 minutes. In further embodiments, a dose of a modified immune cell is administered to the subject over a period from about 20 minutes to about 30 minutes.

In any of the presently disclosed embodiments, a dose comprising a modified immune cell may be administered to the subject intravenously, intratumorally, intrathecally, or into bone marrow.

In still further embodiments, the subject being treated is further receiving immunosuppressive therapy, such as calcineurin inhibitors, corticosteroids, microtubule inhibitors, low dose of a mycophenolic acid prodrug, or any combination thereof. In yet further embodiments, the subject being treated has received a non-myeloablative or a myeloablative hematopoietic cell transplant, wherein the treatment may be administered at least two to at least three months after the non-myeloablative hematopoietic cell transplant.

The level of a CTL immune response may be determined by any one of numerous immunological methods described herein and practiced in the art. The level of a CTL immune response may be determined prior to and following administration of any one of the herein described binding proteins expressed by, for example, a T cell. Cytotoxicity assays for determining CTL activity may be performed using any one of several techniques and methods routinely practiced in the art (see, e.g., Henkart et al., “Cytotoxic T-Lymphocytes” in Fundamental Immunology, Paul (ed.) (2003 Lippincott Williams & Wilkins, Philadelphia, Pa.), pages 1127-50, and references cited therein).

Antigen-specific T cell responses are typically determined by comparisons of observed T cell responses according to any of the herein described T cell functional parameters (e.g., proliferation, cytokine release, CTL activity, altered cell surface marker phenotype, etc.) that may be made between T cells that are exposed to a cognate antigen in an appropriate context (e.g., the antigen used to prime or activate the T cells, when presented by immunocompatible antigen-presenting cells) and T cells from the same source population that are exposed instead to a structurally distinct or irrelevant control antigen. A response to the cognate antigen that is greater, with statistical significance, than the response to the control antigen signifies antigen-specificity. Persistence, spread, antitumor activity, and phenotype of modified immune cells can be determined using markers and assays known in the art and including those described herein (e.g., by flow cytometry gating for a surface-expressed transduction marker that is co-expressed with the binding protein, for a T cell activation or exhaustion marker (e.g., TIM-3, LAG-3, PD-1, TIGT, CD137), by radiation imaging or histology to determine tumor burden, mass, volume, or spread, or the like).

A biological sample may be obtained from a subject for determining the presence and level of an immune response to a binding protein or cell as described herein. A “biological sample” as used herein may be a blood sample (from which serum or plasma may be prepared), biopsy specimen, body fluids (e.g., lung lavage, ascites, mucosal washings, synovial fluid), bone marrow, lymph nodes, tissue explant, tumor, organ culture, or any other tissue or cell preparation from the subject or a biological source. Biological samples may also be obtained from the subject prior to receiving any immunogenic composition, which biological sample is useful as a control for establishing baseline (i.e., pre-immunization) data.

The pharmaceutical compositions described herein may be presented in unit-dose or multi-dose containers, such as infusion bags, sealed ampoules or vials. Such containers may be frozen to preserve the stability of the formulation until use for, e.g., therapy or analysis. The development of suitable dosing and treatment regimens for using the particular compositions described herein in a variety of treatment regimens, including e.g., parenteral or intravenous administration or formulation.

If the subject composition is administered parenterally, the composition may also include sterile aqueous or oleaginous solution or suspension. Suitable non-toxic parenterally acceptable diluents or solvents include water, Ringer's solution, isotonic salt solution, 1,3-butanediol, ethanol, propylene glycol or polythethylene glycols in mixtures with water. Aqueous solutions or suspensions may further comprise one or more buffering agents, such as sodium acetate, sodium citrate, sodium borate or sodium tartrate. Material used in preparing any dosage unit formulation should be pharmaceutically acceptable and substantially non-toxic in the amounts employed. In addition, the active compounds may be incorporated into sustained-release preparation and formulations. Dosage unit form, as used herein, refers to physically discrete units suited as unitary dosages for the subject to be treated; each unit may contain a predetermined quantity of recombinant cells or active compound calculated to produce the desired therapeutic effect in association with an appropriate pharmaceutical carrier.

In general, an appropriate dosage and treatment regimen provides the active molecules or cells in an amount sufficient to provide therapeutic or prophylactic benefit. Such a response can be monitored by establishing an improved clinical outcome (e.g., stable disease, more frequent remissions, complete or partial, or longer disease-free survival) in treated subjects as compared to non-treated subjects. Increases in preexisting immune responses to a tumor protein generally correlate with an improved clinical outcome. Such immune responses may generally be evaluated using imaging techniques for solid tumors or standard proliferation, cytotoxicity or cytokine assays, which are routine in the art and may be performed using samples obtained from a subject before and after treatment.

Methods according to this disclosure may further include administering one or more additional agents to treat the disease or disorder in a combination therapy. For example, in certain embodiments, a combination therapy comprises administering a modified immune cell with (concurrently, simultaneously, or sequentially) an immune checkpoint inhibitor. In some embodiments, a combination therapy comprises administering a modified immune cell with an agonist of a stimulatory immune checkpoint agent. In further embodiments, a combination therapy comprises administering a modified immune cell with a secondary therapy, such as chemotherapeutic agent, a radiation therapy, a surgery, an antibody, or any combination thereof.

As used herein, the term “immune suppression agent” or “immunosuppression agent” refers to one or more cells, proteins, molecules, compounds or complexes providing inhibitory signals to assist in controlling or suppressing an immune response. For example, immune suppression agents include those molecules that partially or totally block immune stimulation; decrease, prevent or delay immune activation; or increase, activate, or up regulate immune suppression. Exemplary immunosuppression agents to target (e.g., with an immune checkpoint inhibitor) include PD-1, PD-L1, PD-L2, CTLA4, TIGIT, LAG3, Tim-3, or any combination thereof.

An immune suppression agent inhibitor (also referred to as an immune checkpoint inhibitor) may be a compound, an antibody, an antibody fragment or fusion polypeptide (e.g., Fc fusion, such as CTLA4-Fc or LAG3-Fc), an antisense molecule, a ribozyme or RNAi molecule, or a low molecular weight organic molecule. In any of the embodiments disclosed herein, a method may comprise administering a modified immune cell with one or more inhibitor of any one of the following immune suppression components, singly or in any combination.

In certain embodiments, a modified immune cell is used in combination with a PD-1 inhibitor, for example a PD-1-specific antibody or binding fragment thereof, such as pidilizumab, nivolumab (Keytruda, formerly MDX-1106), pembrolizumab (Opdivo, formerly MK-3475), MEDI0680 (formerly AMP-514), AMP-224, BMS-936558 or any combination thereof. In further embodiments, binding protein of the present disclosure (or an engineered host cell expressing the same) is used in combination with a PD-L1 specific antibody or binding fragment thereof, such as BMS-936559, durvalumab (MEDI4736), atezolizumab (RG7446), avelumab (MSB0010718C), MPDL3280A, or any combination thereof.

In certain embodiments, a combination therapy comprises a modified immune cell of the present disclosure and a secondary therapy comprising one or more of: an antibody or antigen binding-fragment thereof that is specific for a cancer antigen expressed by the non-inflamed solid tumor, a radiation treatment, a surgery, a chemotherapeutic agent, a cytokine, RNAi, or any combination thereof.

In certain embodiments, a combination therapy method comprises administering a modified immune cell and further administering a radiation treatment or a surgery. Radiation therapy is well-known in the art and includes X-ray therapies, such as gamma-irradiation, and radiopharmaceutical therapies. Surgeries and surgical techniques appropriate to treating a given cancer or non-inflamed solid tumor in a subject are well-known to those of ordinary skill in the art.

In certain embodiments, a combination therapy method comprises administering a modified immune cell and further administering a chemotherapeutic agent. A chemotherapeutic agent includes, but is not limited to, an inhibitor of chromatin function, a topoisomerase inhibitor, a microtubule inhibiting drug, a DNA damaging agent, an antimetabolite (such as folate antagonists, pyrimidine analogs, purine analogs, and sugar-modified analogs), a DNA synthesis inhibitor, a DNA interactive agent (such as an intercalating agent), and a DNA repair inhibitor. Illustrative chemotherapeutic agents include, without limitation, the following groups: anti-metabolites/anti-cancer agents, such as pyrimidine analogs (5-fluorouracil, floxuridine, capecitabine, gemcitabine and cytarabine) and purine analogs, folate antagonists and related inhibitors (mercaptopurine, thioguanine, pentostatin and 2-chlorodeoxyadenosine (cladribine)); antiproliferative/antimitotic agents including natural products such as vinca alkaloids (vinblastine, vincristine, and vinorelbine), microtubule disruptors such as taxane (paclitaxel, docetaxel), vincristin, vinblastin, nocodazole, epothilones and navelbine, epidipodophyllotoxins (etoposide, teniposide), DNA damaging agents (actinomycin, amsacrine, anthracyclines, bleomycin, busulfan, camptothecin, carboplatin, chlorambucil, cisplatin, cyclophosphamide, Cytoxan, dactinomycin, daunorubicin, doxorubicin, epirubicin, hexamethylmelamineoxaliplatin, iphosphamide, melphalan, merchlorehtamine, mitomycin, mitoxantrone, nitrosourea, plicamycin, procarbazine, taxol, taxotere, temozolamide, teniposide, triethylenethiophosphoramide and etoposide (VP 16)); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine (BCNU) and analogs, streptozocin), trazenes-dacarbazinine (DTIC); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory agents; antisecretory agents (breveldin); immunosuppressives (cyclosporine, tacrolimus (FK-506), sirolimus (rapamycin), azathioprine, mycophenolate mofetil); anti-angiogenic compounds (TNP470, genistein) and growth factor inhibitors (vascular endothelial growth factor (VEGF) inhibitors, fibroblast growth factor (FGF) inhibitors); angiotensin receptor blocker; nitric oxide donors; anti-sense oligonucleotides; antibodies (trastuzumab, rituximab); chimeric antigen receptors; cell cycle inhibitors and differentiation inducers (tretinoin); mTOR inhibitors, topoisomerase inhibitors (doxorubicin (adriamycin), amsacrine, camptothecin, daunorubicin, dactinomycin, eniposide, epirubicin, etoposide, idarubicin, irinotecan (CPT-11) and mitoxantrone, topotecan, irinotecan), corticosteroids (cortisone, dexamethasone, hydrocortisone, methylpednisolone, prednisone, and prenisolone); growth factor signal transduction kinase inhibitors; mitochondrial dysfunction inducers, toxins such as Cholera toxin, ricin, Pseudomonas exotoxin, Bordetella pertussis adenylate cyclase toxin, or diphtheria toxin, and caspase activators; and chromatin disruptors.

Cytokines can be used to manipulate host immune response towards anticancer activity. See, e.g., Floros & Tarhini, Semin. Oncol. 42(4):539-548, 2015. Cytokines useful for promoting immune anticancer or antitumor response include, for example, IFN-α, IL-2, IL-3, IL-4, IL-10, IL-12, IL-13, IL-15, IL-16, IL-17, IL-18, IL-21, IL-24, and GM-CSF, singly or in any combination with modified immune cells of this disclosure.

### Therapeutic Selection Criteria

Subjects administered a presently disclosed therapy may, in some embodiments, be evaluated for one or more selection criteria in order to receive the therapy (e.g., a first and/or second dose of a modified immune cell); e.g., to increase the likelihood that the therapy will be safely tolerated, efficacious, or both. For example, in some embodiments comprising administration of a first and a second dose of a modified immune cell to a patient having a cancer (e.g., NSCLC or TNBC), at least about 21 days after administering the first dose and prior to administering the second dose: (i) the subject exhibits a stable or a progressive cancer, as described herein; (ii) the subject has not experienced a toxicity event (e.g., cytokine release syndrome (characterized by, for example, fever, fatigue, hypotension, tachycardia, nausea, capillary leak, or cardiac, renal, anorexia, or hepatic dysfunction); neurological toxicity (characterized by, for example, confusion, delirium, aphasia or seizure); on-target, off-tumor toxicity wherein the modified immune cell kills non-target cells that express the antigen (such as, for example, B-cell aplasia in CAR-T cell therapies targeting CD19 or CD20); anaphylaxis or allergy to the modified immune cell or binding protein thereof; or insertional oncogenesis; see, e.g., Bonifant et al., Mol. Ther.—Oncolytics 3, 16011, doi:10.1038/mto.2016.11 (2016), the cell therapy-associated toxicities and management techniques of which are incorporated herein by reference in their entirety); (iii) the modified immune cells that were administered in the first dose are detected in a sample obtained from the subject (e.g., in serum, peripheral blood (including PBMCs), a tumor biopsy, or the like); or (iv) any combination of (i)-(iii). Modified immune cells can be detected using any appropriate technique; e.g., by use of detectably labeled antibodies specific for a modified immune cell protein, such as the binding protein or a cell surface expression marker that is associated with expression of the binding protein, or by RNA or DNA sequencing.

In some embodiments, a stable cancer comprises: (a) no statistically significant change in a number of tumors as compared to the number of tumors present prior to administering the first dose (e.g., as determined using MRI, PET, CT, ultrasound, radionuclide, multimodal imaging, and the like); (b) no statistically significant change in tumor size or volume as compared to the tumor size or volume prior administration of the first dose; (c) no spread or metastasis of the cancer to another organ and/or tissue as compared to the organs and/or tissues prior to administration of the first dose; or (d) any combination of (a)-(c).

In certain embodiments, a progressive cancer comprises (a) a statistically significant change in a number of tumors as compared to the number of tumors prior to administration of the first dose; (b) a statistically significant change in a tumor size or volume as compared to the tumor size or volume prior to administration of the first dose; (c) a spread or metastasis of the cancer to another organ and/or tissue as compared to the organs and/or tissues prior to administration of the first dose; or (d) any combination of (a)-(c).

In some embodiments, a toxicity event comprises a severe neurotoxicity event (e.g., a seizure, loss of speech, loss of muscle function, significant cognitive impairment), severe cytokine release syndrome (sCRS; e.g., grade ≥3 organ toxicity with highly elevated levels of cytokines such as IFN-γ, GM-CSF, IL-10, or IL-6), or both.

In certain embodiments, the subject is selected for treatment according to one or more criteria as set forth in Table 1 or Table 2, below.

TABLE 1
Inclusion Criteria

Subjects with non-small cell lung cancer that is metastatic or inoperable and
who have been treated with at least one line of prior therapy or declined
conventional therapy
Subjects with known epidermal growth factor receptor (EGFR) or anaplastic
lymphoma kinase (ALK) mutations must have been treated on at least one
line of molecularly targeted therapy (e.g., erlotinib, crizotinib)
Subjects must have measurable disease by at least one of criteria (i) or (ii)
below:

| | |
|---|---|
| (i) | Extra skeletal disease that can be accurately measured in at least one |
| | dimension as >= 10 mm with conventional computed tomography |
| | (CT) techniques as defined by Response Evaluation Criteria in Solid |
| | Tumors (RECIST) 1.1 |
| (ii) | Skeletal or bone-only disease measurable by fludeoxyglucose F 18 |
| | (FDG) positron emission tomography (PET) imaging |

ROR1 expression in >20% of the primary tumor or metastasis by
immunohistochemistry (IHC)
Karnofsky performance status of >=70%
Subjects must be off chemotherapy for a minimum of 3 weeks prior to start of
treatment; targeted therapies must be stopped at least 3 days prior to start of
lymphodepletion
Negative pregnancy test for women of childbearing potential; subjects of
childbearing potential are those who have not been surgically sterilized or
have not been free from menses for >1 year
Fertile male and female subjects must be willing to use a contraceptive
method before, during and for at least two months after the T cell infusion
Ability to understand and provide informed consent

TABLE 2
Inclusion Criteria

Histologically confirmed diagnosis of metastatic TNBC; i.e. breast cancer that
is estrogen receptor (ER) negative (=<10%), progesterone receptor (PR)
negative (=<10%), and human epidermal growth factor receptor 2 (HER2)
negative (0 or 1+ by immunohistochemistry or negative for gene
amplification by fluorescence in situ hybridization [FISH])
Subjects must have measurable disease by at least one of criteria (i) or (ii)
below:

| | |
|---|---|
| (i) | Extra skeletal disease that can be accurately measured in at least one |
| | dimension as >=10 mm with conventional computed tomography |
| | (CT) techniques as defined by Response Evaluation Criteria in Solid |
| | Tumors (RECIST) 1.1 |
| (ii) | Skeletal or bone-only disease measurable by fludeoxyglucose F 18 |
| | (FDG) positron emission tomography (PET) imaging |

Subjects must have received standard adjuvant, neoadjuvant, and/or
metastatic chemotherapy per National Comprehensive Cancer Network
(NCCN) or institutional practice; no maximum on number of prior systemic
treatment regimens
Subjects may receive agents to protect against skeletal-related complications
such as zoledronic acid or denosumab
ROR1 expression in >20% of the primary tumor or metastasis by IHC
Karnofsky performance status of >=70%
Subjects must be off chemotherapy for a minimum of 3 weeks prior to
planned leukapheresis
Negative pregnancy test for women of childbearing potential; subjects of
childbearing potential are those who have not been surgically sterilized or
have not been free from menses for >1 year
Fertile male and female subjects must be willing to use a contraceptive
method before, during and for at least two months after the T cell infusion
Ability to understand and provide informed consent

In some embodiments, the cancer has metastasized in the subject prior to administration of the first dose of the modified T cell.

### EXAMPLES

### Example 1

### Phase I Clinical Study of Immunotherapy Using Anti-ROR1 CAR-T Cells

### Objectives

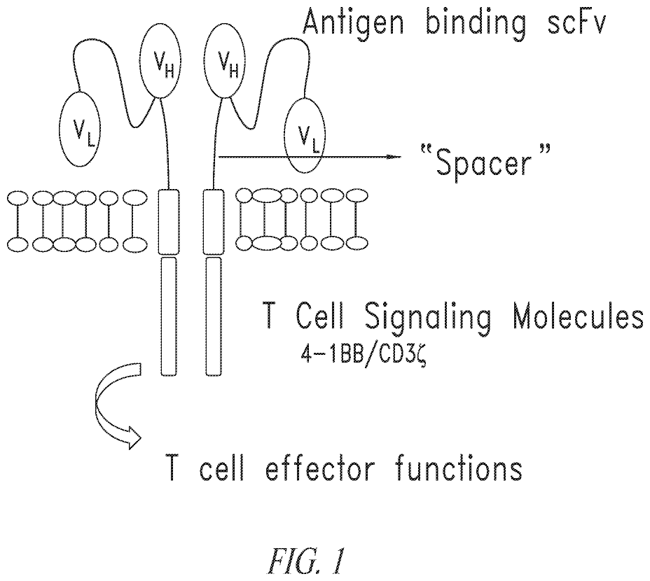

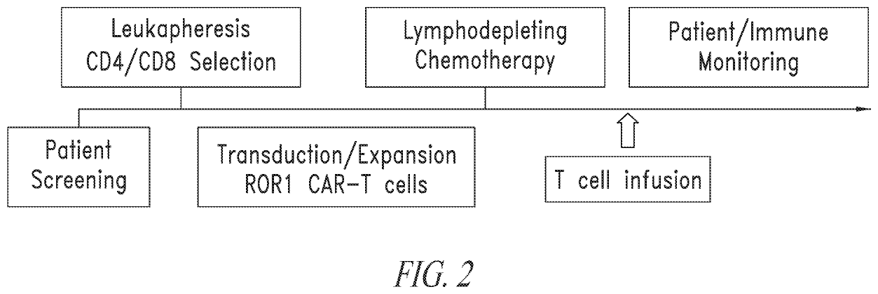

ROR1 is a type 1 transmembrane tyrosine kinase receptor that plays a critical role in embryonic and fetal development. ROR1 has been described as a possible oncogene and is expressed in numerous malignancies (see, e.g., Balakrishnan et al, Clin. Cancer Res., 23:12 (2017). A single-center study was conducted to evaluate the safety and anti-tumor activity of adoptively transferred autologous CAR-T cells targeting the ROR1 antigen in patients with advanced ROR1+ chronic lymphocytic leukemia (CLL), mantle cell lymphoma (MCL), or acute lymphoblastic leukemia ALL (“Cohort A”) and in patients with ROR1+ non small-cell lung cancer (NSCLC) or triple negative breast cancer (TNBC; not expressing the genes for estrogen receptor (ER), progesterone receptor (PR), and HER2/Neu)) (“Cohort B”) in a phase 1 dose escalation trial (see FIG. 2). The primary study objective was evaluating safety of the CAR-T cell product (ex vivo expanded 1:1 CD4+:CD8+ autologous T cells transduced with a lentiviral vector encoding a ROR1-specific CAR that includes a scFv from antibody R12, a spacer domain, and 4-1BB and CD3t signaling domains, as described in PCT Publication No. WO 2014/031687, represented schematically in FIG. 1). Secondary objectives were: determining duration of in vivo persistence and phenotype of ROR1 CAR-T cells; trafficking of ROR1 CAR-T cells traffic to tumor site and function in vivo; and determining anti-tumor activity of ROR1 CAR-T cells in patients with measurable tumor burden by RECIST 1.1 (see Eisenhauer et al., Eur. J. Cancer 45:228-247 (2009)).

### Selection Criteria

Briefly, patients were selected using the criteria set forth in Tables 3 (Cohort A), 4 (Cohort B NSCLC), and 5 (Cohort B TNBC):

TABLE 3
Inclusion Criteria for Cohort A Patients

CLL patients who are beyond first remission and who have failed combination
chemoimmunotherapy with regimens containing a purine analogue and anti-
CD20 antibody, or who have failed tyrosine kinase or phosphatidylinositol 3
(PI3) kinase inhibitors, or who were not eligible for or declined such therapy;
patients with fludarabine refractory disease are eligible
MCL patients who are beyond first remission and previously treated with
chemoimmunotherapy; patients who have relapsed following autologous
hematopoietic cell transplant (HCT) are eligible
ALL patients who have relapsed or have residual disease following treatment
with curative intent; ALL patients must have ROR1 expressed on >90% of the
leukemia blasts to be eligible
Confirmation of diagnosis by internal pathology review of initial or subsequent
biopsy or other pathologic material at the clinical treatment site
Evidence of ROR1 expression by immunohistochemistry or flow cytometry on
any prior or current tumor specimen
Karnofsky performance status >=70%
Negative pregnancy test for women of childbearing potential; subjects of
childbearing potential are those who have not been surgically sterilized or have
not been free from menses for >1 year
Fertile male and female patients must be willing to use a contraceptive method
before, during, and for at least two months after the T cell infusion
Ability to understand and provide informed consent

TABLE 4
Inclusion Criteria for Cohort B NSCLC Patients

| Patients with non-small cell lung cancer that is metastatic or inoperable and | |
|---|---|
| who have been treated with at least one line of prior therapy or declined | |
| conventional therapy | |
| Patients with known epidermal growth factor receptor (EGFR) or anaplastic | |
| lymphoma kinase (ALK) mutations must have been treated on at least one | |
| line of molecularly targeted therapy (e.g., erlotinib, crizotinib) | |
| Patients must have measurable disease by at least one of criteria (i) or (ii) | |
| below: | |
| (iii) | Extra skeletal disease that can be accurately measured in at least one |
| | dimension as >=10 mm with conventional computed tomography |
| | (CT) techniques as defined by Response Evaluation Criteria in Solid |
| | Tumors (RECIST) 1.1 |
| (iv) | Skeletal or bone-only disease measurable by fludeoxyglucose F 18 |
| | (FDG) positron emission tomography (PET) imaging |

ROR1 expression in >20% of the primary tumor or metastasis by
immunohistochemistry (IHC)
Karnofsky performance status of >=70%
Patients must be off chemotherapy for a minimum of 3 weeks prior to start of
treatment; targeted therapies must be stopped at least 3 days prior to start of
lymphodepletion
Negative pregnancy test for women of childbearing potential; subjects of
childbearing potential are those who have not been surgically sterilized or
have not been free from menses for >1 year
Fertile male and female patients must be willing to use a contraceptive
method before, during and for at least two months after the T cell infusion
Ability to understand and provide informed consent

TABLE 5
Inclusion Criteria for Cohort B TNBC Patients

Histologically confirmed diagnosis of metastatic TNBC; i.e. breast cancer that
is estrogen receptor (ER) negative (=<10%), progesterone receptor (PR)
negative (=<10%), and human epidermal growth factor receptor 2 (HER2)
negative (0 or 1+ by immunohistochemistry or negative for gene
amplification by fluorescence in situ hybridization [FISH])
Patients must have measurable disease by at least one of criteria (i) or (ii)
below:

| | |
|---|---|
| (iii) | Extra skeletal disease that can be accurately measured in at least one |
| | dimension as >=10 mm with conventional computed tomography |
| | (CT) techniques as defined by Response Evaluation Criteria in Solid |
| | Tumors (RECIST) 1.1 |
| (iv) | Skeletal or bone-only disease measurable by fludeoxyglucose F 18 |
| | (FDG) positron emission tomography (PET) imaging |

Patients must have received standard adjuvant, neoadjuvant, and/or metastatic
chemotherapy per National Comprehensive Cancer Network (NCCN) or
institutional practice; no maximum on number of prior systemic treatment
regimens
Patients may receive agents to protect against skeletal-related complications
such as zoledronic acid or denosumab
ROR1 expression in >20% of the primary tumor or metastasis by IHC
Karnofsky performance status of >=70%
Patients must be off chemotherapy for a minimum of 3 weeks prior to planned
leukapheresis
Negative pregnancy test for women of childbearing potential; subjects of
childbearing potential are those who have not been surgically sterilized or
have not been free from menses for >1 year
Fertile male and female patients must be willing to use a contraceptive
method before, during and for at least two months after the T cell infusion
Ability to understand and provide informed consent

### Leukapheresis

Next, leukapharesis was performed on each patient using standard operating procedures for obtaining peripheral blood mononuclear cells (PBMCs).

### Production of CAR-T Cell Product

The PBMCs were sorted to isolate CD8+ and CD4+ cells. PMBCs were divided into two aliquots and enriched for T cells (one aliquot enriched for CD8+ T cells; the other aliquot for CD4+) T cells using clinical-grade reagents and SOPs developed in-house.

The enriched fractions were independently cultured with anti-CD3/anti-CD28 beads and IL-2, and transduced with a lentiviral vector encoding the ROR1 CAR and a truncated EGFR (“EGFRt”) cell surface marker of transduction. The CAR-T cell product was formulated in a 1:1 ratio (50%+/−15%) of CD4+ and CD8+ CAR-T cells. Prior to release for patient infusion, cell product was tested in vitro for transgene expression, minimum dosage (5×10⁴ mg/kg EGFRt+), and release criteria including absence of endotoxin, gram staining, sterility, mycoplasma, RCL, and viability.

### Lymphodepletion Therapy

Prior to the first infusion of ROR1 CAR-T cells, patients received cytoreductive and/or lymphodepleting chemotherapy including cyclophosphamide to provide lymphodepletion, to facilitate T cell survival, and to reduce the tumor burden prior to infusion of ROR1 CAR-T cells. For some treatments, a combination regime of cyclophosphamide (300 mg/m²) and fludarabine (30 mg/m²) were used. For others, oxaliplatin and cyclophosphamide were used.

### Administration of the CAR-T Cell Product

The CAR-T cell product was then administered to patients approximately 36 to 96 hours after conclusion of lymphodepleting chemotherapy. On the day of scheduled T cell infusion, patients underwent a clinical evaluation and a clinical determination for appropriateness to proceed with T cell administration.

Dose levels (DLs) as follows were administered intravenously over 20-30 minutes, with DL1 as the starting dose for both Cohorts:

TABLE 6
Dose Levels of ROR1 CAR-T Cells

| Dose Level | Cell Dose, EGFRt+ cells/kg |
|---|---|
| 0 | Up to 1 × 10⁵ |
| 1 | Up to 3.3 × 10⁵ |
| 2 | Up to 1 × 10⁶ |
| 3 | Up to 3.3 × 10⁶ |
| 4 | Up to 1 × 10⁷ |

### Dose Escalation/De-Escalation

Dose escalation or de-escalation was determined by a continual reassessment of toxicity. Treatment of patients in the dose-escalation/de-escalation groups were staggered with a minimum of a 21-day interval following infusion between each patient and 35 days before escalation to the next dose level. Patients received a second infusion of ROR1 CAR-T cells with or without additional lymphodepletion chemotherapy at the same dose level (for those that received the highest cell dose) or up to the next highest dose level if, after the first infusion, there was persistent disease, there were no toxicities attributed to the first infusion, the patient was at least 21 days from the first T cell infusion, and there were no clinical and/or laboratory exclusion criteria.

Persistence of CAR-T cells in blood, cytokine levels, measures of immunogenicity and multi-parametric flow cytometry were evaluated at multiple time points. Imaging assessments by RECIST 1.1 was performed at day 28-90, then at 6 and 12 months, and every 6 months, as clinically indicated.

### Example 2

### Safety and Antitumor Activity of RorOR1 CAR-T Cells

To date, seven (10) patients (7 TNBC, 3 NSCLC) were enrolled and received treatment. Disease indications, age, number of prior post-metastatic therapies, and metastatic sites of the patients are shown in Table 7.

TABLE 7
Patient Characteristics

| | | | # of prior | |
|---|---|---|---|---|
| | | | metastatic | |
| Subject | Indication | Age | therapies | Metastatic Site(s) |
| X461 | NSCLC | — | 9 | Thoracic nodes, anterior chest |
| | | | | wall, pancreas, CNS (treated) |
| X475 | TNBC | 38 | 9 | Lungs, thoracic nodes, |
| | | | | abd/retroperitoneal nodes, CNS |
| | | | | (treated) |
| X501 | NSCLC | — | 3 | Lungs, thoracic nodes |
| X552 | TNBC | 59 | 11 | Liver (>30 lesions), bones |
| X566 | TNBC | 67 | 4 | Left SCV, left axilla, parasternal |
| | | | | chest wall, sternum |
| X579 | TNBC | 51 | 3 | Liver |
| X590 | NSCLC | — | 5 | Lung |
| X641 | TNBC | 27 | 4 | Left lung, left pre-pectoral node, |
| | | | | sternum |
| X650 | TNBC | 35 | 4 | Diffuse bone mets |
| X668 | TNBC | 61 | 10 | Liver, diffuse bone mets |

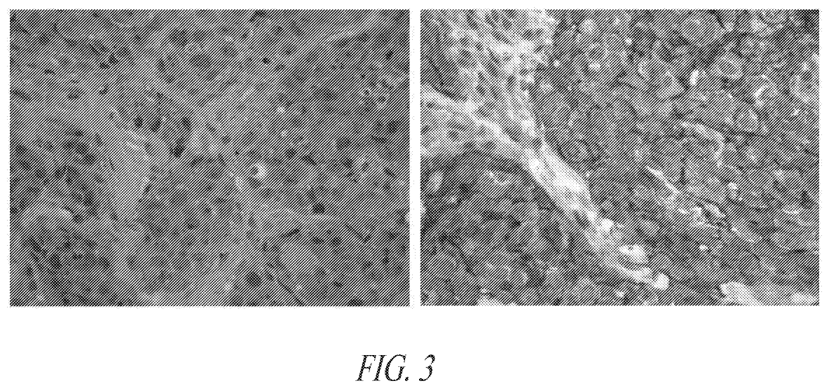

From these patients, all 34 TNBC tumors and 11 of 20 NSCLC tumors screened had >20% ROR1 expression as determined by IHC (representative data from patient X566 is shown in FIG. 3). Eight (8) patients (6 TNBC, 2 NSCLC) were evaluable for response. Table 8 shows ROR1 CAR-T cell dose levels, toxicity events and grade, response (RECIST 1.1) of the indicated patients at first post-infusion assessment, as well as whether the patients were subsequently re-dosed with the CAR-T cells.

TABLE 8
CAR-T dose levels, lymphodepleting chemotherapy,
toxicity events, response, and re-treatment

| | | | Lympho- | | | |
|---|---|---|---|---|---|---|
| | | Dose | depleting | CRS, | Best Response | |
| Subject | Indication | Level | chemotherapy | grade | (~day 30) | Redose |
| X461 | NSCLC | 1 | — | Yes, 1 | Stable disease | No |
| X475 | TNBC | 1 | Flu/Cy | No | Progressive | No |
| | | | | | disease | |
| X501 | NSCLC | 2 | — | No | Stable disease | Yes |
| X552 | TNBC | 2 | Flu/Cy; Ox/Cy | Yes, 1 | Stable disease | Yes |
| X566 | TNBC | 2 | Ox/Cy; Flu/Cy | No | Stable disease, | Yes |
| | | | | | PR after 2^{nd} | |
| | | | | | infusion† | |
| X579 | TNBC | 2* | Ox/Cy | Yes, 1 | Stable disease | No |
| X590 | NSCLC | 2* | — | Not | Not evaluable | — |
| | | | | evaluable | | |
| X641 | TNBC | 3* | Ox/Cy | Yes, 3 | Progressive | No |
| | | | | | disease | |
| X650 | TNBC | 3* | Ox/Cy | Yes, 1 | Progressive | No |
| | | | | | disease with | |
| | | | | | clinical response | |
| X668 | TNBC | 3 | Ox/Cy | Yes, 1 | Not evaluable | No |

Flu = Fludarabine.
Cy = Cyclophosphamide.
Ox = Oxaliplatin.
CRS = Cytokine Release Syndrome.
†= partial response at Day 28 following second CAR-T cell infusion.
*= Target cell dose was not achieved for CD8 fraction of a ~1:1 CD4:CD8 CAR-T cell product.

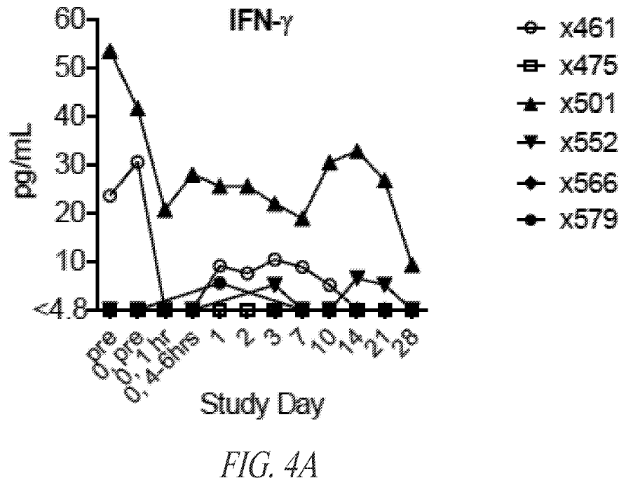

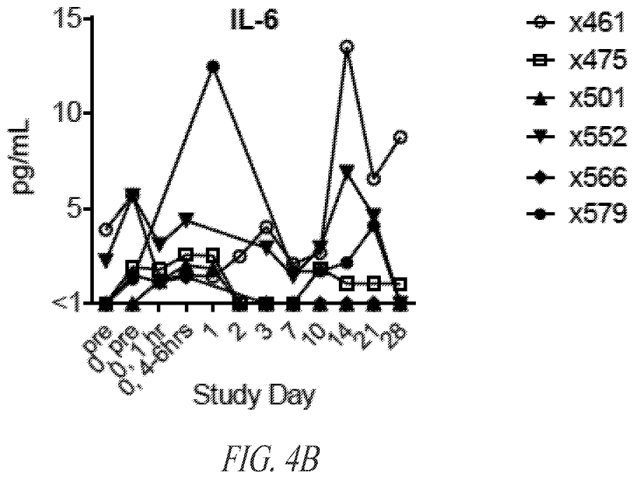

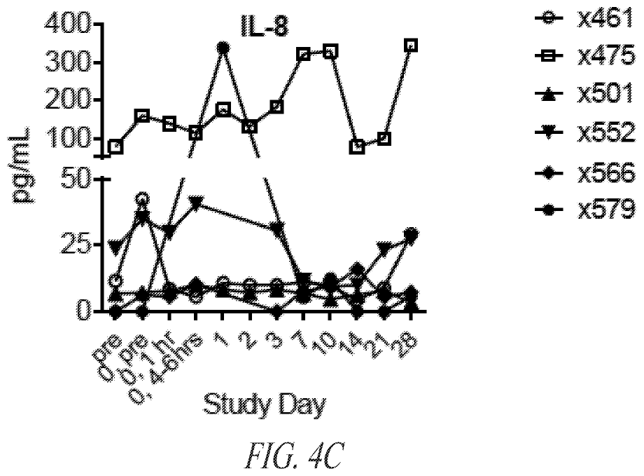

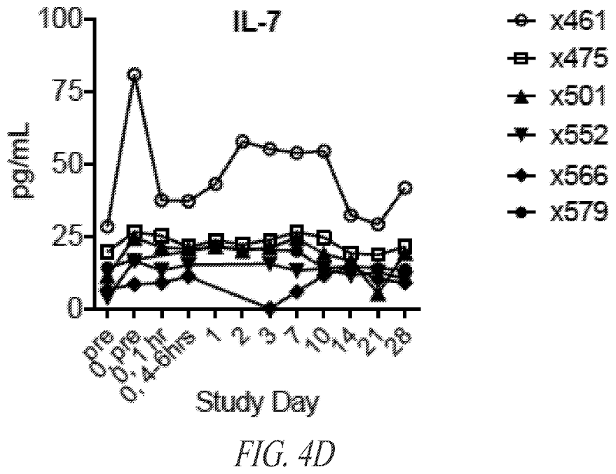

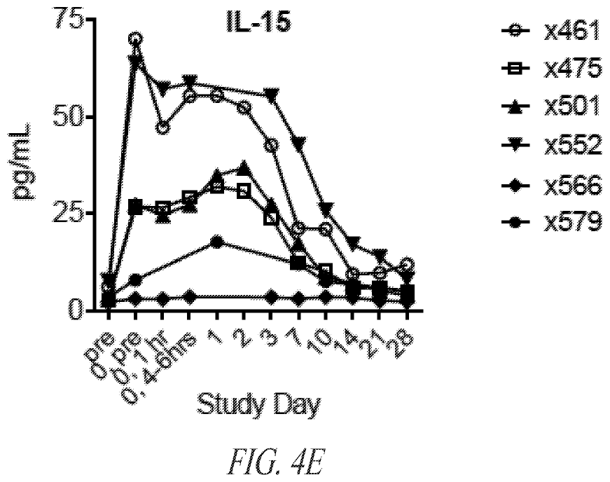

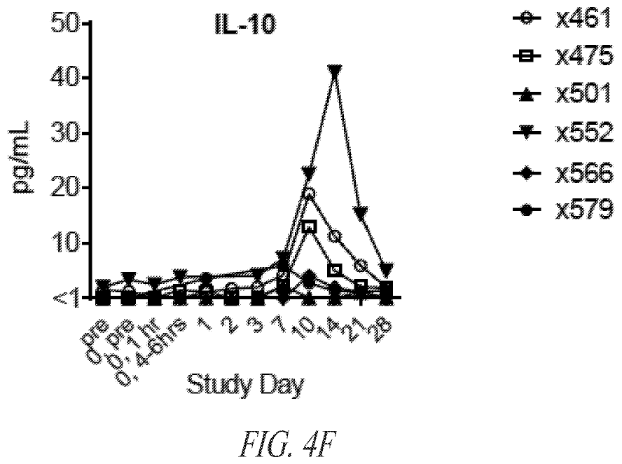

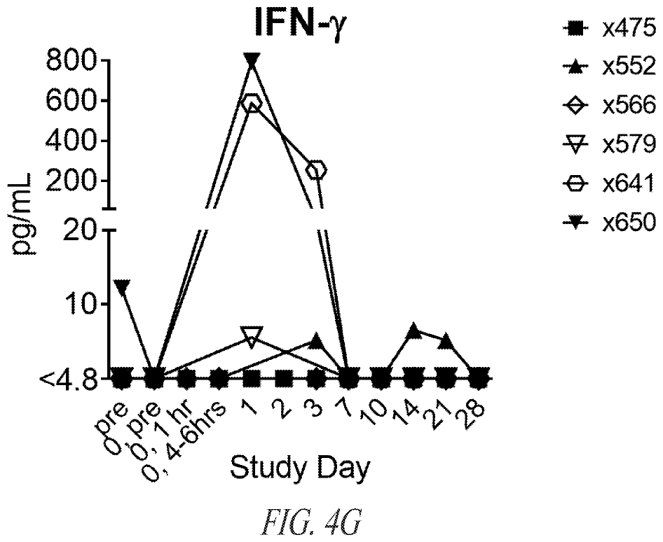

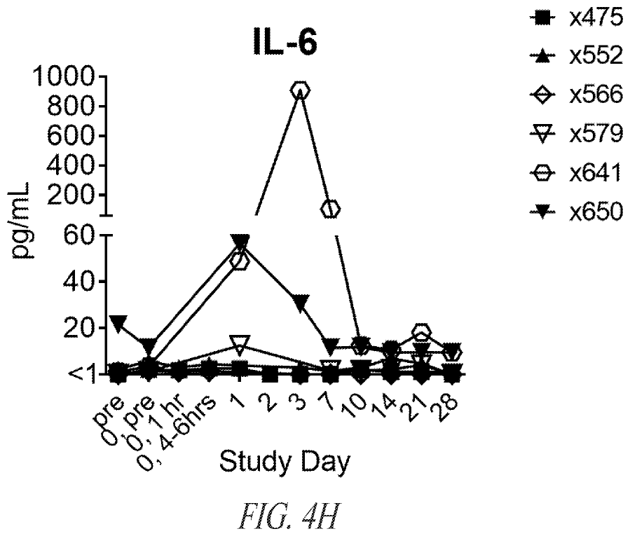

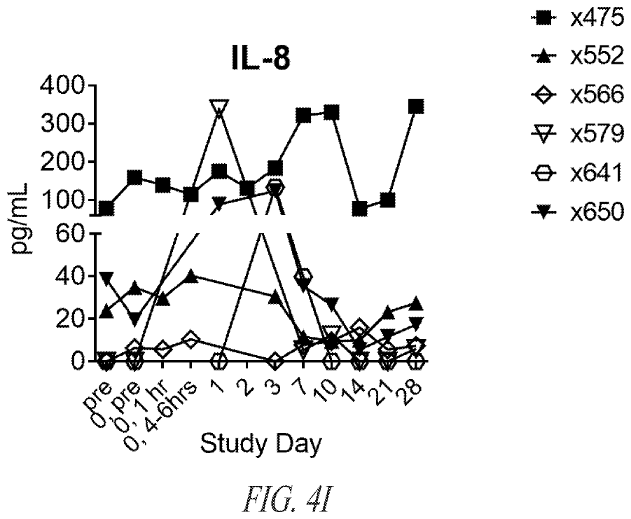

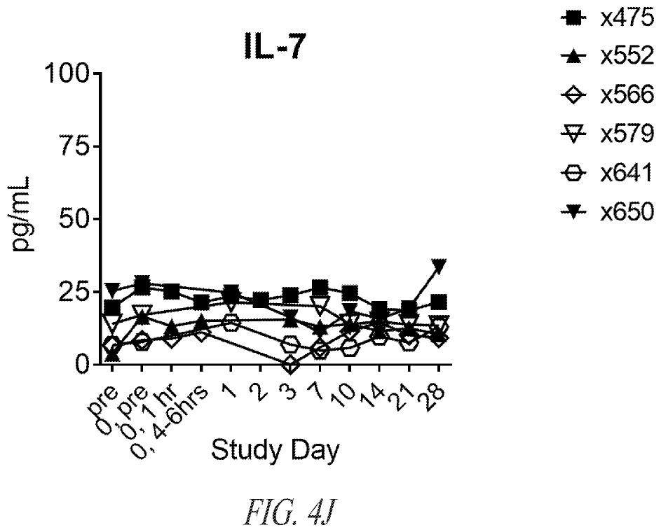

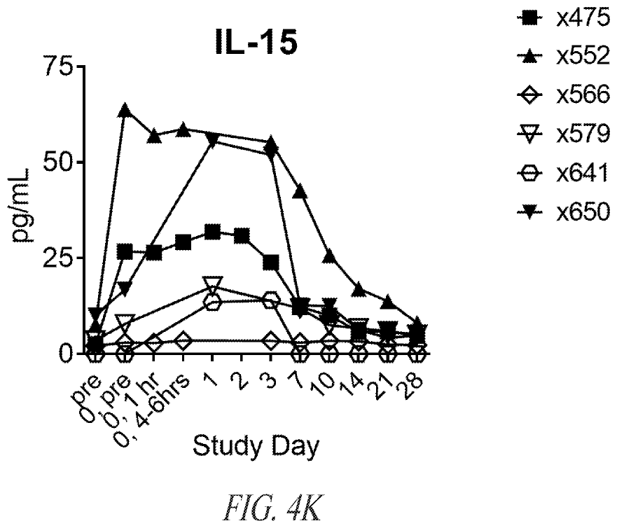

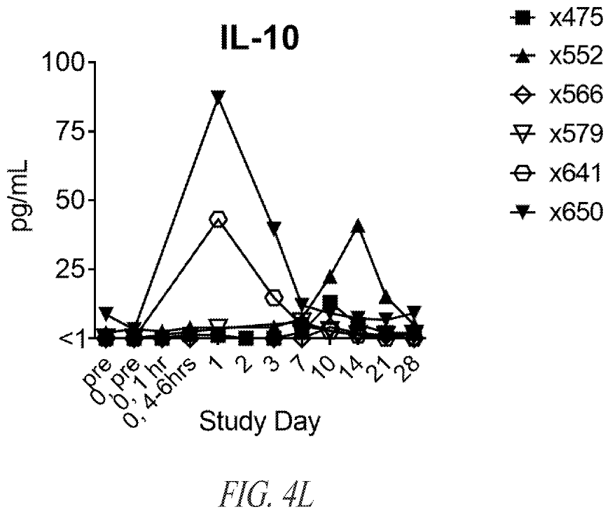

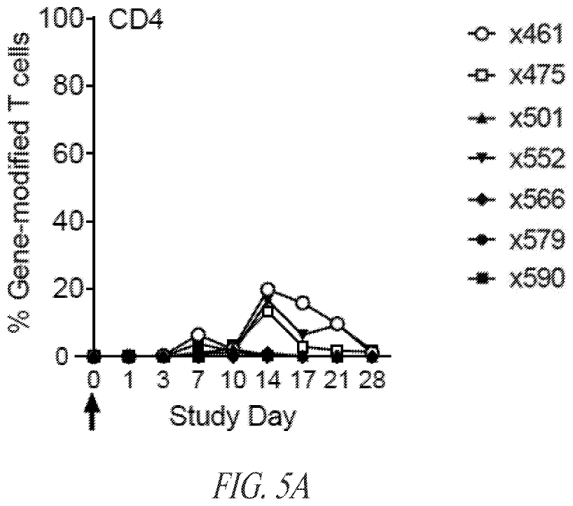

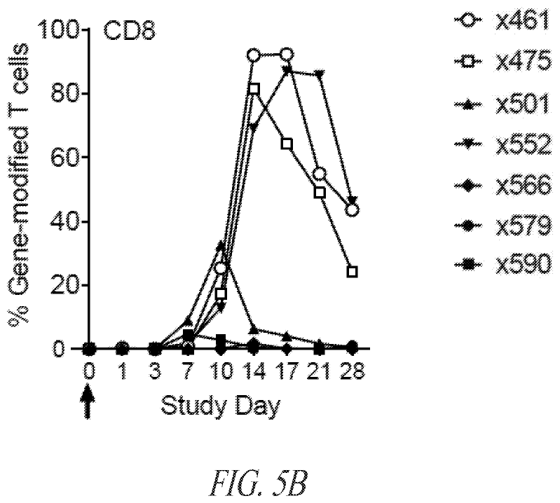

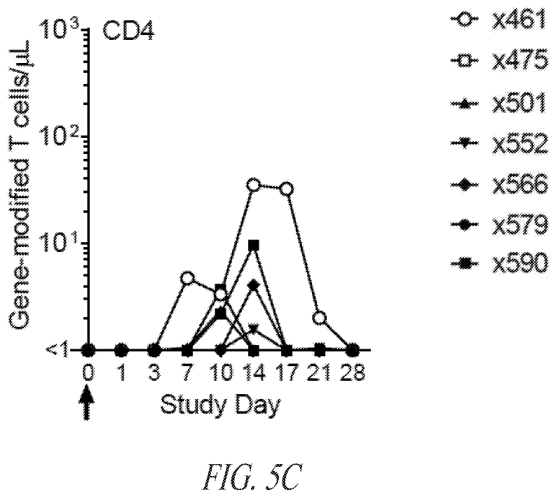

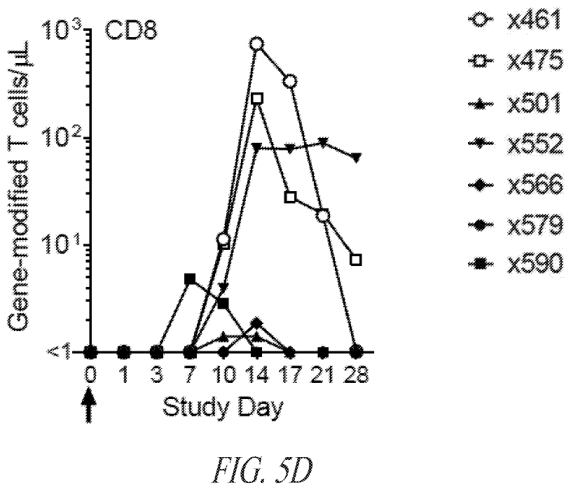

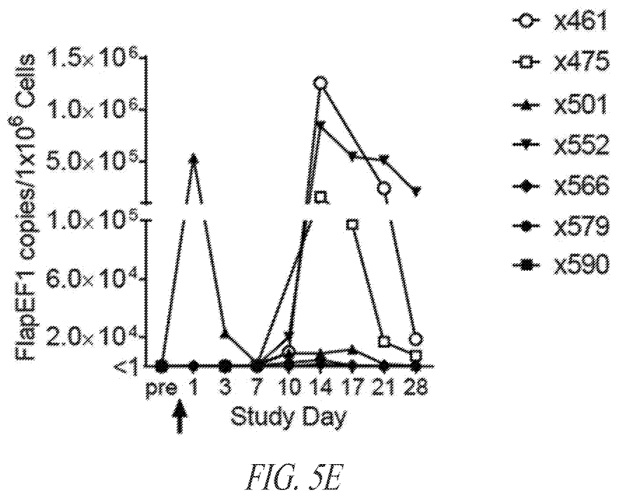

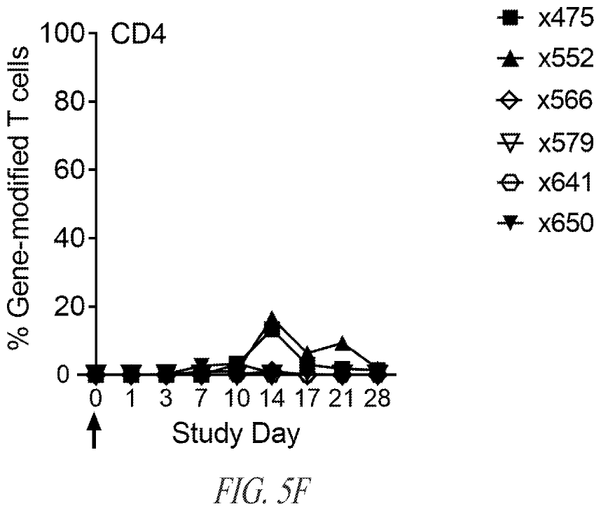

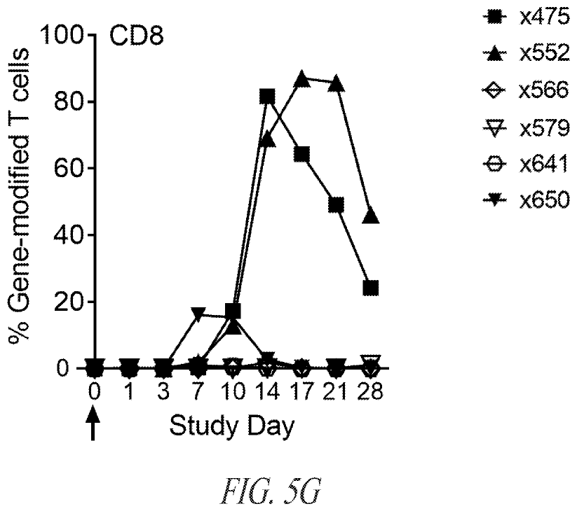

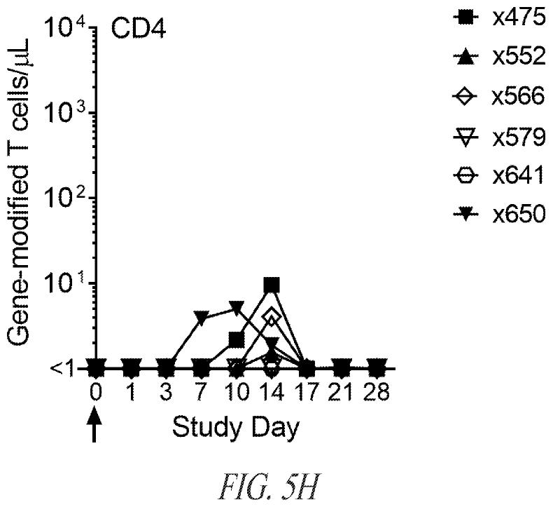

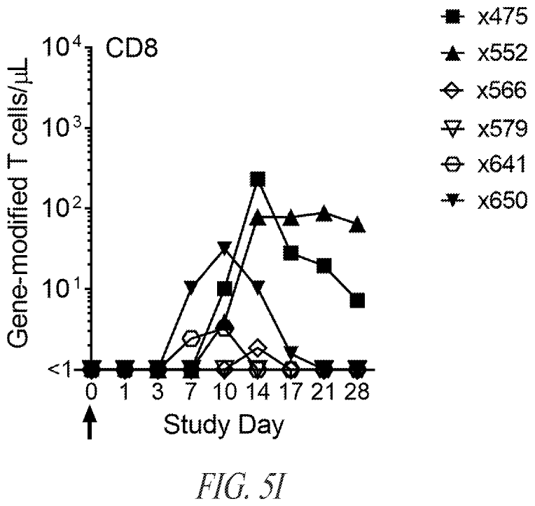

Serum cytokine profiles after first ROR1 CAR-T cell infusion were determined by Luminex cytokine assay; data are shown in FIGS. 4A-4L. No dose-limiting toxicities, severe neurotoxicity or severe cytokine release syndrome events (sCRS) were observed at dose levels 1 (3.3×10⁵ cells) and 2 (1×10⁶ cells). Three patients experienced grade 1 CRS. No infusion reactions or tumor lysis syndrome (TLS) have been observed in patients treated to date. One patient experienced grade 3 CRS responsive to dexamethasone, tocilizumab. As a secondary outcome measure, CAR-T cell persistence was determined by flow cytometry (FIGS. 5A-5D, 5F-5I) and qPCR (FIGS. 5E and 5J). Three patients had evidence of CAR-T cell expansion between days 14 and 20, with peak CD8+ [CAR-T] up to 747/uL.

Four patients (2 NSCLC; 2 TNBC) demonstrated a mixed response with decreased disease burden at some metastatic sites. One patient (X566) achieved a partial response (PR) with 45% decrease in the sum of the longest diameter of target lesions as measured on Day +28 following the second CAR-T cell infusion.

These data show that ROR1 CAR-T cells are safely transferred at doses of up to 1×10⁶ cells, expand in vivo in patients with NSCLC and TNBC, and possess antitumor activity.

### Example 3

### Further Analysis of ROR1 CAR-T Cell Therapy

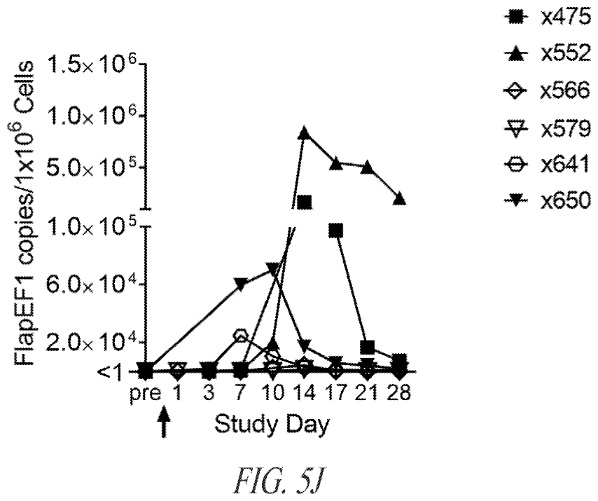

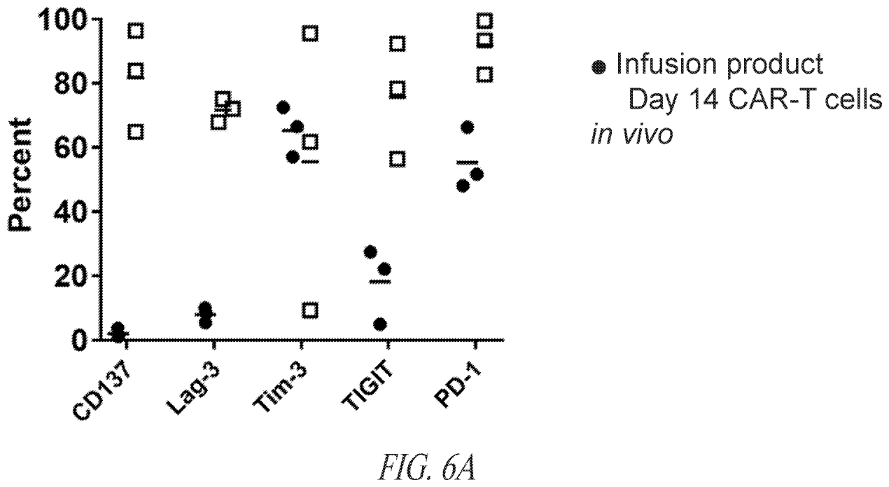

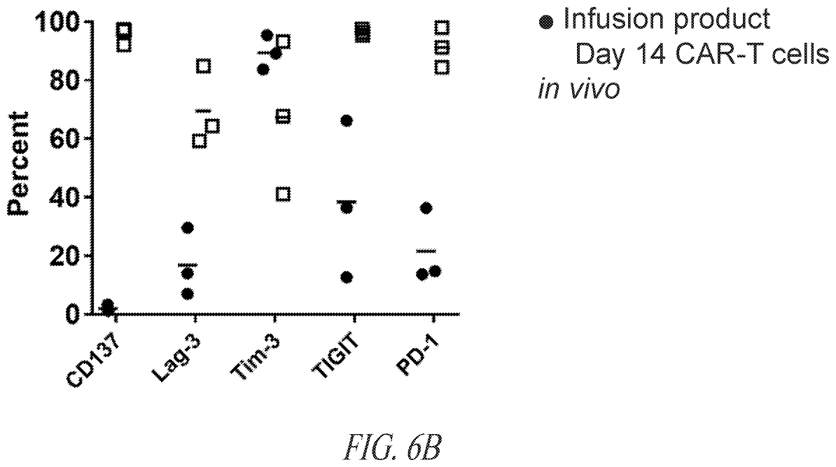

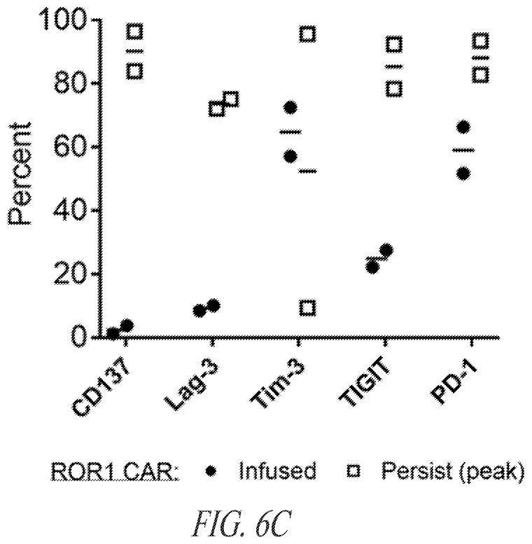

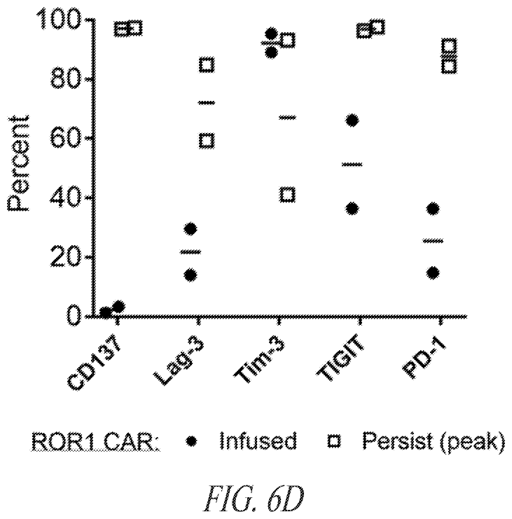

The ability of transferred disease-targeting T cells to maintain function in vivo is believed to be important for long-term therapeutic benefit. T cell exhaustion has been observed in some cancers and infections, and is believed to involve signaling by a number of T cell inhibitory proteins. See, e.g., Yi et al., Immunology 129(4):474 (2010). To determine the functionality of ROR1 CAR-T cells following transfer, expression of activation and exhaustion markers on both CD4+(FIGS. 6A and 6C) and CD8+(FIGS. 6B and 6D) cells was measured in the infusion product and at Day 14 following transfer. Antibody staining for the markers revealed upregulation of inhibitory receptors on CAR-T cells at the peak of expansion, which was confirmed by RNA sequencing.

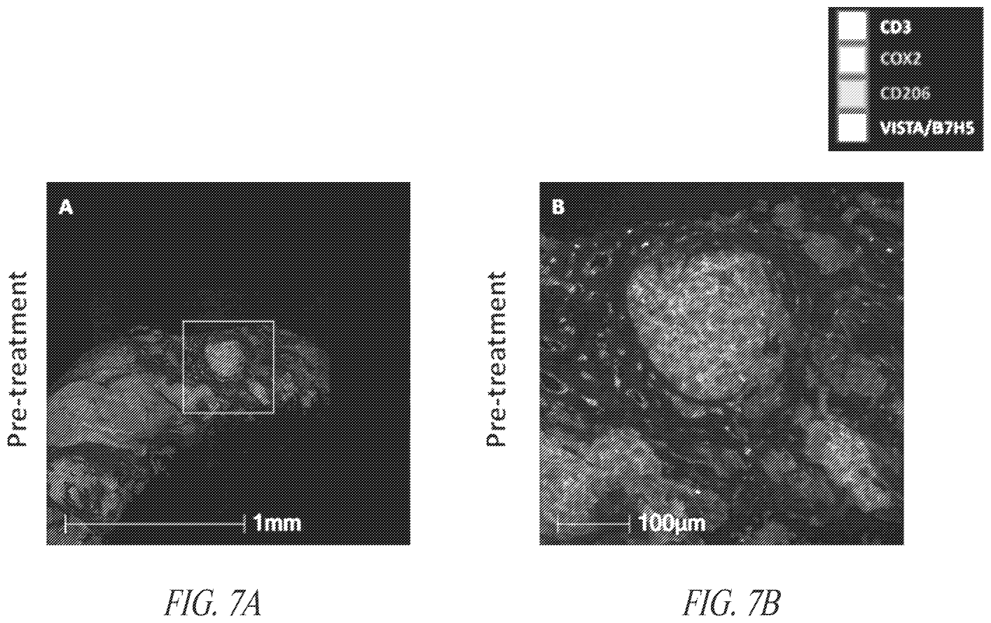

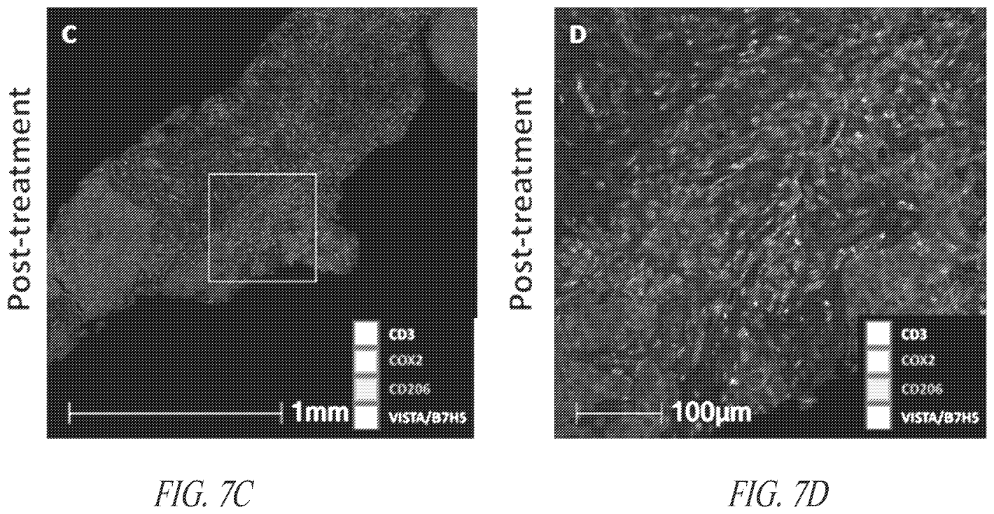

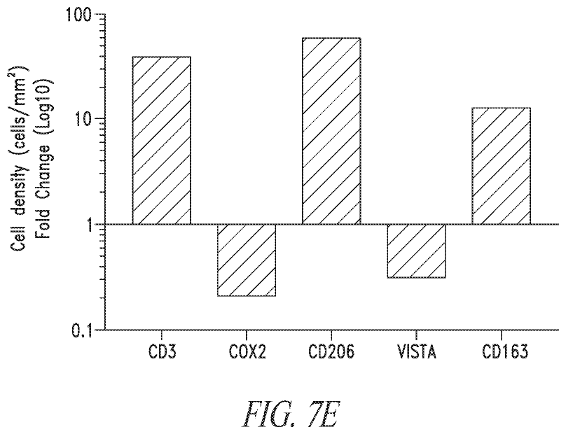

Next, post-treatment tumor biopsies were performed in two patients (data for patient X566 shown in FIGS. 7A-7D) to evaluate immune cell infiltration following CAR-T cell therapy. As shown by IHC staining in FIGS. 7A-7D, an influx of CD3+ T cells and macrophages into the tumor was observed following therapy with the ROR1 CAR-T cells. T cell infiltration in solid tumors has been observed to correlate with tumor control and improved patient outcomes. See, e.g., Lanitis et al., Annals of Oncology, 28 supp 12:xiii8-xiii32 (2017).

Further studies are performed to understand and address mechanisms that affect homing, persistence, and/or function of CAR-T cells at tumor sites. Additional eligible patients are enrolled with dose-escalation.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, including U.S. Provisional Patent Application No. 62/657,642, filed Apr. 13, 2018, are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

What is claimed is:

1. A method for treating cancer in a subject, the method comprising administering to the subject a first dose of a modified T cell comprising a heterologous polynucleotide that encodes a binding protein, wherein the encoded binding protein includes:
(a) an extracellular component comprising a binding domain that specifically binds to a ROR1 antigen, wherein the binding domain comprises
(i) a variable heavy chain (VH) region comprising or consisting of the amino acid sequence set forth in SEQ ID NO:7 and a variable light chain (VL) region comprising or consisting of the amino acid sequence set forth in SEQ ID NO:8 and/or
(ii) a CDRL1 amino acid sequence according to SEQ ID NO:4, a CDRL2 amino acid sequence according to SEQ ID NO:5, a CDRL3 amino acid sequence according to SEQ ID NO:6, a CDRH1 amino acid sequence according to SEQ ID NO:1, a CDRH2 amino acid sequence according to SEQ ID NO:2, and a CDRH3 amino acid sequence according to SEQ ID NO:3;
(b) an intracellular component comprising all or a portion of a 4-1BB signaling domain and all or a portion of a CD3t signaling domain; and
(c) a transmembrane domain disposed between the extracellular component and the intracellular component, wherein the transmembrane domain comprises all or a portion of a CD8 transmembrane domain or a CD28 transmembrane domain,
wherein the first dose comprises at least about 3.3×10⁵ of the modified T cell/kg, and
wherein the subject has previously been administered a first lymphodepleting chemotherapy comprising cyclophosphamide from about 36 to about 96 hours prior to being administered the first dose,
thereby treating the cancer in the subject.

2. The method of claim 1, wherein the first dose comprises at least about 1×10⁶ of the modified T cell/kg.

3. The method of claim 1 or 2, wherein the first dose comprises at least about 3.3×10⁶ of the modified T cell/kg.

4. The method of any one of claims 1-3, wherein the first dose comprises at least about 1×10⁷ of the modified T cell/kg.

5. The method of any one of claims 1-4, wherein the first lymphodepleting chemotherapy further comprises fludarabine.

6. The method of any one of claims 1-5, wherein the first lymphodepleting chemotherapy further comprises oxaliplatin.

7. The method of any one of claims 1-6, wherein the modified T cell comprises a modified CD8+ T cell, a modified CD4+ T cell, or both.

8. The method of claim 7, wherein the first dose comprises modified CD8+ T cells and modified CD4+ T cells in about a 1:1 ratio.

9. The method of any one of claims 1-8, wherein the binding domain comprises a single chain variable fragment (scFv).

10. The method of claim 9, wherein the scFv comprises a VH domain, a VL domain, and a linker disposed between the VH domain and the VL domain.

11. The method of claim 10, wherein the scFv comprises a VL-linker-VH orientation or a VH-linker-VL orientation.

12. The method of any one of claims 1-11, wherein the extracellular domain further comprises a spacer region disposed between the binding domain and the transmembrane domain.

13. The method of claim 12, wherein the spacer region comprises all or a portion of an immunoglobulin hinge or a modified version thereof, at least a portion of a CH2, and/or at least a portion of a CH3.

14. The method of claim 13, wherein the immunoglobulin hinge or a modified version thereof comprises an IgG hinge or a modified version thereof.

15. The method of claim 14, wherein the IgG hinge or a modified version thereof comprises an IgG1, IgG2, IgG3, or IgG4 hinge or a modified version thereof.

16. The method of any one of claims 1-15, further comprising administering to the subject a second dose of the modified T cell.

17. The method of claim 16, wherein the second dose comprises a same amount of the modified T cell as compared to the first dose.

18. The method of claim 16, wherein the second dose comprises a reduced amount of the modified T cell as compared to the first dose.

19. The method of claim 16, second dose comprises an increased amount of the modified T cell as compared to the first dose.

20. The method of claim 19, wherein the first dose comprises about 1×10⁶ of the modified T cell/kg and the second dose comprises about 3.3×10⁶ of the modified T cell/kg.

21. The method of claim 19, wherein the first dose comprises about 3.3×10⁶ of the modified T cell/kg and the second dose comprises about 1.0×10⁷ of the modified T cell/kg.

22. The method of any one of claims 16-21, wherein the second dose is administered at least about 28 days after the first dose.

23. The method of any one of claims 16-22, further comprising, following administration of the first dose of the modified T cell and prior to administration of the second dose of the modified T cell, administering to the subject a second lymphodepleting chemotherapy.

24. The method of any one of claims 1-23, wherein either or both of the first dose and the second dose of the modified T cell is administered to the subject over a period from about 1 minute to about 1 hour.

25. The method of claim 24, wherein the first dose of the modified T cell, the second dose of the modified T cell, or both, is administered to the subject over a period from about 20 minutes to about 30 minutes.

26. The method of any one of claims 1-25, wherein either or both of the first dose and the second dose is administered to the subject intravenously, intratumorally, intrathecally, or into bone marrow.

27. The method of any one of claims 1-26, wherein the cancer is selected from the group consisting of:
(a) a solid tumor, wherein the solid tumor is selected the group consisting of triple negative breast cancer (TNBC) and non-small-cell lung cancer (NSCLC); and
(b) a hematological malignancy, wherein the hematological malignancy is selected from the group consisting of acute lymphoblastic leukemia (ALL), chronic lymphocytic leukemia (CLL), and mantle cell lymphoma (MCL).

28. The method of any one of claims 16-27, wherein, at least about 21 days after administering the first dose of the modified T cell and prior to administering the second dose of the modified T cell:
(i) the subject exhibits a stable or a progressive cancer;
(ii) the subject has not experienced a toxicity event;
(v) the modified T cells administered in the first dose are detected in a sample obtained from the subject; or
(vi) any combination of (i)-(iii).

29. The method of claim 28, wherein the stable cancer comprises:
(a) no statistically significant change in a number of tumors as compared to the number of tumors prior to administration of the first dose;
(b) no statistically significant change in tumor size or volume as compared to the tumor size or volume prior to administration of the first dose;
(c) no spread or metastasis of the cancer to another organ and/or tissue as compared to the organs and/or tissues prior to administration of the first dose; or
(d) any combination of (a)-(c).

30. The method of claim 28, wherein the progressive cancer comprises:
(a) a statistically significant change in a number of tumors as compared to the number of tumors prior to administration of the first dose;
(b) a statistically significant change in a tumor size or volume as compared to the tumor size or volume prior to administration of the first dose;
(c) a spread or metastasis of the cancer to another organ and/or tissue as compared to the organs and/or tissues prior to administration of the first dose; or
(d) any combination of (a)-(c).

31. The method of any one of claims 28-30, wherein the toxicity event comprises a severe neurotoxicity event, severe cytokine release syndrome (CRS), or both.

32. The method of any one of claims 28-31, wherein the sample from the subject comprises blood or peripheral blood mononuclear cells (PBMCs).

33. The method of any one of claims 28-32, wherein the sample from the subject comprises a tumor or a tumor biopsy.

34. The method of any one of claims 1-33, wherein the subject is selected for treatment according to one or more criteria as set forth in Table 1 or Table 2.

35. The method of any one of claims 1-34, wherein the cancer is recurrent.

36. The method of any one of claims 1-35, wherein the cancer has metastasized in the subject prior to administration of the first dose of the modified T cell.

37. The method of any one of claims 1-36, further comprising administering to the subject an agent that specifically binds to and/or is an inhibitor of an immune suppression component.

38. The method of claim 37, wherein the inhibitor of the immune suppression component is selected from an inhibitor of PD-1, PD-L1, CTLA4, TIGIT, Tim-3, LAG3, or any combination thereof.

39. The method of claim 37 or 38, wherein the inhibitor of the immune suppression component is selected from the group consisting of an antibody or antigen binding fragment thereof a binding protein; a small molecule; an RNAi molecule; a ribozyme; an aptamer; an antisense oligonucleotide; or any combination thereof.

40. The method of any one of claims 37-39, wherein the inhibitor of the immune suppression component comprises pidilizumab, nivolumab, pembrolizumab, MEDI0680, AMP-224, BMS-936558 BMS-936559, durvalumab, atezolizumab, avelumab, MPDL3280A, ipilimumab, tremelimumab, abatacept, belatacept, BMS-986207, OMP-313M32, RG-6058, AB-154, COM-902, Monoclonal Antibodies to Antagonize TIGIT for Oncology (315293), Monoclonal Antibody to Antagonize TIGIT for Oncology (328189), Monoclonal Antibody to Antagonize TIGIT for Oncology (350426), ENUM-009 (326504), or Monoclonal Antibodies to Antagonize TIGIT for Oncology (331672), MK-7684, or an antigen binding fragment thereof, or any combination thereof.

41. The method of any one of claims 1-40, further comprising administering to the subject a therapeutically effective amount of an agonist of a stimulatory immune checkpoint molecule.

42. The method of claim 41, wherein the agonist is selected from urelumab, MEDI6469, MEDI6383, MEDI0562, lenalidomide, pomalidomide, CDX-1127, TGN1412, CD80, CD86, CP-870,893, rhuCD40L, SGN-40, IL-2, GSK3359609, mAb 88.2, JTX-2011, Icos 145-1, Icos 314-8, or any combination thereof.

43. The method of any one of claims 1-42, further comprising administering to the subject a secondary therapy selected from an antibody or antigen-binding fragment specific for a cancer antigen, a chemotherapeutic agent, surgery, radiation therapy, an anticancer cytokine, an RNA interference molecule, or any combination thereof.

44. The method of claim 43, wherein the secondary therapy comprises a cytokine selected from IFN-γ, IFN-α, IL-2, IL-3, IL-4, IL-10, IL-12, IL-13, IL-15, IL-16, IL-17, IL-18, IL-21, IL-24, GM-CSF, or any combination thereof.


SEQUENCE LISTING (70 sequences)

SEQ ID NO: 1 - protein, 5 aa, Artificial Sequence
  Synthetic sequence R12 antibody CDRH1
AYYMS

SEQ ID NO: 2 - protein, 17 aa, Artificial Sequence
  Synthetic sequence R12 antibody CDRH2
TIYPSSGKTYYATWVNG

SEQ ID NO: 3 - protein, 12 aa, Artificial Sequence
  Synthetic sequence R12 antibody CDRH3
DSYADDGALFNI

SEQ ID NO: 4 - protein, 12 aa, Artificial Sequence
  Synthetic sequence R12 antibody CDRL1
TLSSAHKTDTID

SEQ ID NO: 5 - protein, 7 aa, Artificial Sequence
  Synthetic sequence R12 antibody CDRL2
GSYTKRP

SEQ ID NO: 6 - protein, 9 aa, Artificial Sequence
  Synthetic sequence R12 antibody CDRL3
GADYIGGYV

SEQ ID NO: 7 - protein, 121 aa, Artificial Sequence
  Synthetic sequence R12 antibody VH
QEQLVESGGRLVTPGGSLTLSCKASGFDFSAYYMSWVRQAPGKGLEWIATIYPSSGKTYYATWVNGRFTISSDNAQNTVDLQMNSLTAADRATYFCARDSYADDGALFNIWGPGTLVTISS

SEQ ID NO: 8 - protein, 112 aa, Artificial Sequence
  Synthetic sequence R12 antibody VL
ELVLTQSPSVSAALGSPAKITCTLSSAHKTDTIDWYQQLQGEAPRYLMQVQSDGSYTKRPGVPDRFSGSSSGADRYLIIPSVQADDEADYYCGADYIGGYVFGGGTQLTVTG

SEQ ID NO: 9 - protein, 248 aa, Artificial Sequence
  Synthetic sequence R12 scFv
QEQLVESGGRLVTPGGSLTLSCKASGFDFSAYYMSWVRQAPGKGLEWIATIYPSSGKTYYATWVNGRFTISSDNAQNTVDLQMNSLTAADRATYFCARDSYADDGALFNIWGPGTLVTISSGGGGSGGGGSGGGGSELVLTQSPSVSAALGSPAKITCTLSSAHKTDTIDWYQQLQGEAPRYLMQVQSDGSYTKRPGVPDRFSGSSSGADRYLIIPSVQADDEADYYCGADYIGGYVFGGGTQLTVTG

SEQ ID NO: 10 - protein, 5 aa, Artificial Sequence
  Synthetic sequence 2A2 antibody CDRH1
DYEMH

SEQ ID NO: 11 - protein, 17 aa, Artificial Sequence
  Synthetic sequence 2A2 antibody CDRH2
AIDPETGGTAYNQKFKG

SEQ ID NO: 12 - protein, 9 aa, Artificial Sequence
  Synthetic sequence 2A2 antibody CDRH3
YYDYDSFTY

SEQ ID NO: 13 - protein, 11 aa, Artificial Sequence
  Synthetic sequence 2A2 antibody CDRL1
KASQNVDAAVA

SEQ ID NO: 14 - protein, 7 aa, Artificial Sequence
  Synthetic sequence 2A2 antibody CDRL2
SASNRYT

SEQ ID NO: 15 - protein, 9 aa, Artificial Sequence
  Synthetic sequence 2A2 antibody CDRL3
QQYDIYPYT

SEQ ID NO: 16 - protein, 118 aa, Artificial Sequence
  Synthetic sequence 2A2 antibody VH
QVQLQQSGAELVRPGASVTLSCKASGYTFSDYEMHWVIQTPVHGLEWIGAIDPETGGTAYNQKFKGKAILTADKSSSTAYMELRSLTSEDSAVYYCTGYYDYDSFTYWGQGTLVTVSA

SEQ ID NO: 17 - protein, 107 aa, Artificial Sequence
  Synthetic sequence 2A2 antibody VL
DIVMTQSQKIMSTTVGDRVSITCKASQNVDAAVAWYQQKPGQSPKLLIYSASNRYTGVPDRFTGSGSGTDFTLTISNMQSEDLADYFCQQYDIYPYTFGGGTKLEIK

SEQ ID NO: 18 - protein, 237 aa, Artificial Sequence
  Synthetic sequence 2A2 scFv
QVQLQQSGAELVRPGASVTLSCKASGYTFSDYEMHWVIQTPVHGLEWIGAIDPETGGTAYNQKFKGKAILTADKSSSTAYMELRSLTSEDSAVYYCTGYYDYDSFTYWGQGTLVTVSAGGGSGGGSGGGSDIVMTQSQKIMSTTVGDRVSITCKASQNVDAAVAWYQQKPGQSPKLLIYSASNRYTGVPDRFTGSGSGTDFTLTISNMQSEDLADYFCQQYDIYPYTFGGGTKLEIK

SEQ ID NO: 19 - protein, 5 aa, Artificial Sequence
  Synthetic sequence R11 antibody CDRH1
DYPIS

SEQ ID NO: 20 - protein, 16 aa, Artificial Sequence
  Synthetic sequence R11 antibody CDRH2
FINSGGSTWYASWVKG

SEQ ID NO: 21 - protein, 11 aa, Artificial Sequence
  Synthetic sequence R11 antibody CDRH3
GYSTYYGDFNI

SEQ ID NO: 22 - protein, 11 aa, Artificial Sequence
  Synthetic sequence R11 antibody CDRL1
QASQSIDSNLA

SEQ ID NO: 23 - protein, 7 aa, Artificial Sequence
  Synthetic sequence R11 antibody CDRL2
RASNLAS

SEQ ID NO: 24 - protein, 12 aa, Artificial Sequence
  Synthetic sequence R11 antibody CDRL3
LGGVGNVSYRTS

SEQ ID NO: 25 - protein, 116 aa, Artificial Sequence
  Synthetic sequence R11 antibody VH
QSVKESEGDLVTPAGNLTLTCTASGSDINDYPISWVRQAPGKGLEWIGFINSGGSTWYASWVKGRFTISRTSTTVDLKMTSLTTDDTATYFCARGYSTYYGDFNIWGPGTLVTISS

SEQ ID NO: 26 - protein, 110 aa, Artificial Sequence
  Synthetic sequence R11 antibody VL
ELVMTQTPSSTSGAVGGTVTINCQASQSIDSNLAWFQQKPGQPPTLLIYRASNLASGVPSRFSGSRSGTEYTLTISGVQREDAATYYCLGGVGNVSYRTSFGGGTEVVVK

SEQ ID NO: 27 - protein, 241 aa, Artificial Sequence
  Synthetic sequence R11 scFv
QSVKESEGDLVTPAGNLTLTCTASGSDINDYPISWVRQAPGKGLEWIGFINSGGSTWYASWVKGRFTISRTSTTVDLKMTSLTTDDTATYFCARGYSTYYGDFNIWGPGTLVTISSGGGGSGGGGSGGGGSELVMTQTPSSTSGAVGGTVTINCQASQSIDSNLAWFQQKPGQPPTLLIYRASNLASGVPSRFSGSRSGTEYTLTISGVQREDAATYYCLGGVGNVSYRTSFGGGTEVVVK

SEQ ID NO: 28 - protein, 5 aa, Artificial Sequence
  Synthetic sequence Y31 antibody CDRL1
SHWMS

SEQ ID NO: 29 - protein, 16 aa, Artificial Sequence
  Synthetic sequence Y31 antibody CDRH2
IIAASGSTYYANWAKG

SEQ ID NO: 30 - protein, 12 aa, Artificial Sequence
  Synthetic sequence Y31 antibody CDRH3
DYGDYRLVTFNI

SEQ ID NO: 31 - protein, 11 aa, Artificial Sequence
  Synthetic sequence Y31 antibody CDRL1
QASQSIGSYLA

SEQ ID NO: 32 - protein, 7 aa, Artificial Sequence
  Synthetic sequence Y31 antibody CDRL2
YASNLAS

SEQ ID NO: 33 - protein, 9 aa, Artificial Sequence
  Synthetic sequence Y31 antibody CDRL3
LGSLNSDNV

SEQ ID NO: 34 - protein, 6 aa, Artificial Sequence
  Synthetic sequence anti-ROR1 scFv CDRH1
DTIDWY

SEQ ID NO: 35 - protein, 16 aa, Artificial Sequence
  Synthetic sequence anti-ROR1 scFv CDRH2
VQSDGSYTKRPGVPDR

SEQ ID NO: 36 - protein, 8 aa, Artificial Sequence
  Synthetic sequence anti-ROR1 scFv CDRH3
YIGGYVFG

SEQ ID NO: 37 - protein, 11 aa, Artificial Sequence
  Synthetic sequence anti-ROR1 scFv CDRL1
ASGFDFSAYYM

SEQ ID NO: 38 - protein, 7 aa, Artificial Sequence
  Synthetic sequence anti-ROR1 scFv CDRL2
TIYPSSG

SEQ ID NO: 39 - protein, 9 aa, Artificial Sequence
  Synthetic sequence anti-ROR1 scFv CDRL3
ADRATYFCA

SEQ ID NO: 40 - protein, 5 aa, Artificial Sequence
  Synthetic sequence anti-ROR1 scFv CDRH1
SNLAW

SEQ ID NO: 41 - protein, 16 aa, Artificial Sequence
  Synthetic sequence anti-ROR1 scFv CDRH2
RASNLASGVPSRFSGS

SEQ ID NO: 42 - protein, 8 aa, Artificial Sequence
  Synthetic sequence anti-ROR1 scFv CDRH3
NVSYRTSF

SEQ ID NO: 43 - protein, 11 aa, Artificial Sequence
  Synthetic sequence anti-ROR1 scFv CDRL1
SGSDINDYPIS

SEQ ID NO: 44 - protein, 7 aa, Artificial Sequence
  Synthetic sequence anti-ROR1 scFv CDRL2
INSGGST

SEQ ID NO: 45 - protein, 8 aa, Artificial Sequence
  Synthetic sequence anti-ROR1 scFv CDRL3
YFCARGYS

SEQ ID NO: 46 - protein, 12 aa, Artificial Sequence
  Synthetic sequence amino acid spacer
ESKYGPPCPPCP

SEQ ID NO: 47 - protein, 12 aa, Artificial Sequence
  Synthetic sequence amino acid spacer
ESKYGPPCPSCP

SEQ ID NO: 48 - protein, 15 aa, Artificial Sequence
  Synthetic sequence amino acid spacer
GGGGSGGGGSGGGGS

SEQ ID NO: 49 - protein, 327 aa, Artificial Sequence
  Synthetic sequence IgG4 Fc
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 50 - protein, 18 aa, Artificial Sequence
  Synthetic sequence Thosea asigna virus 2A (T2A) self-cleaving element
EGRGSLLTCGDVEENPGP

SEQ ID NO: 51 - protein, 21 aa, Artificial Sequence
  Synthetic sequence T2A, modified
GSGEGRGSLLTCGDVEENPGP

SEQ ID NO: 52 - protein, 19 aa, Artificial Sequence
  Synthetic sequence Porcine teschovirus-1 2A (P2A) self-cleaving element
ATNFSLLKQAGDVEENPGP

SEQ ID NO: 53 - protein, 22 aa, Artificial Sequence
  Synthetic sequence P2A, modified
GSGATNFSLLKQAGDVEENPGP

SEQ ID NO: 54 - protein, 21 aa, Artificial Sequence
  Synthetic sequence Equine rhinitis A virus 2A (E2A) self-cleaving element
QCTNYALLKLAGSDVESNPGP

SEQ ID NO: 55 - protein, 24 aa, Artificial Sequence
  Synthetic sequence E2A, modified
GSGQCTNYALLKLAGSDVESNPGP

SEQ ID NO: 56 - protein, 22 aa, Artificial Sequence
  Synthetic sequence foot-and-mouth disease virus 2A (F2A) self-cleaving element
VKQTLNFDLLKLAGDVESNPGP

SEQ ID NO: 57 - protein, 25 aa, Artificial Sequence
  Synthetic sequence T2A, modified
GSGVKQTLNFDLLKLAGDVESNPGP

SEQ ID NO: 58 - DNA, 25 bp, Artificial Sequence
  Synthetic sequence TRAC_sgRNA_pLenti_F1, nucleotide
caccggagaatcaaaatcggtgaat

SEQ ID NO: 59 - DNA, 25 bp, Artificial Sequence
  Synthetic sequence TRAC_sgRNA_pLenti_R1
aaacattcaccgattttgattctcc

SEQ ID NO: 60 - DNA, 24 bp, Artificial Sequence
  Synthetic sequence PD1_sgRNA_F1
caccgcagttgtgtgacacggaag

SEQ ID NO: 61 - DNA, 24 bp, Artificial Sequence
  Synthetic sequence PD1_sgRNA_R1
aaaccttccgtgtcacacaactgc

SEQ ID NO: 62 - DNA, 25 bp, Artificial Sequence
  Synthetic sequence CTLA4_sgRNA_F1
caccggcaaaggtgagtgagacttt

SEQ ID NO: 63 - DNA, 25 bp, Artificial Sequence
  Synthetic sequence CTLA4_sgRNA_R1
aaacaaagtctcactcacctttgcc

SEQ ID NO: 64 - DNA, 25 bp, Artificial Sequence
  Synthetic sequence LAG3_sgRNA_F1
caccggtttctgcagccgctttggg

SEQ ID NO: 65 - DNA, 25 bp, Artificial Sequence
  Synthetic sequence LAG3_sgRNA_R2
aaaccccaaagcggctgcagaaacc

SEQ ID NO: 66 - DNA, 72 bp, Artificial Sequence
  Synthetic sequence
ctcgagggcggcggagagggcagaggaagtcttctaacatgcggtgacgtggaggagaatcccggccctagg

SEQ ID NO: 67 - protein, 20 aa, Artificial Sequence
  Synthetic seqeunce
  VARIANT (2)...(10): any one or all of amino acids 2-10 can either be present or absent.
  VARIANT (12)...(20): any one or all of amino acids 12-20can either be present or absent.
GGGGGGGGGGSSSSSSSSSS

SEQ ID NO: 68 - protein, 10 aa, Artificial Sequence
  Synthetic seqeunce
GGGGSGGGGS

SEQ ID NO: 69 - protein, 8 aa, Artificial Sequence
  Synthetic seqeunce
GGGSGGGS

SEQ ID NO: 70 - protein, 11 aa, Artificial Sequence
  Synthetic seqeunce
GGGSGGGSGGS